(19) United States
(12) Patent Application Publication
(10) Pub. No.: US 2026/0035324 A1
(43) Pub. Date: Feb. 5, 2026

(54) RECYCLED CONTENT ORGANIC CHEMICAL COMPOUNDS FROM WASTE PLASTIC

(71) Applicant: Eastman Chemical Company, Kingsport, TN (US)

(72) Inventors: Nick Allen Collins, Fall Branch, TN (US); Daryl Bitting, Longview, TX (US); Michael Gary Polasek, Richmond, TX (US); Xianchun Wu, Longview, TX (US); David Eugene Slivensky, Tatum, TX (US)

(73) Assignee: Eastman Chemical Company, Kingsport, TN (US)

(21) Appl. No.: 19/100,018

(22) PCT Filed: Jul. 20, 2023
(86) PCT No.: PCT/US2023/070541

§ 371 Date: Jan. 30, 2025

Related U.S. Application Data
(60) Provisional application No. 63/370,259, filed on Aug. 3, 2022.

(51) Int. Cl. C07C 13/18; C07C 15/04; C07C 15/06; C07C 55/14; C07D 223/02; C08G 69/00; C10G 55/04; C10G 55/06

(52) U.S. Cl. CPC C07C 13/18; C07C 15/04; C07C 15/06; C07C 55/14; C07D 223/02; C08G 69/00; C10G 55/04; C10G 55/06; C10G 2300/1003; C10G 2400/02; C10G 2400/30

(57) ABSTRACT

Processes and facilities for producing several types of recycled content organic chemical compounds from waste plastic. Processing schemes are described herein for converting waste plastic (or hydrocarbon having recycled content derived from waste plastic) into useful intermediate chemicals and final products. In some aspects, recycled content aromatics (r-aromatics) can be processed to provide recycled content benzene (r-benzene) and/or recycled content toluene (r-toluene), which can be further processed to form a variety of intermediate and final organic chemical compounds including, but not limited to, recycled content nylons, recycled content polystyrene. recycled content benzoic acid, and recycled content phenol.

### BACKGROUND

Aromatic compounds such as benzene, toluene, and xylenes are important industrial chemicals used in a variety of applications. Each of these compounds is used to form a variety of chemical intermediates, such as dicarboxylic acids and esters, which can be used in the production of several different types of polymers, including polyesters, nylon, and others. Most conventional production routes for these materials utilize fossil fuel-derived feeds. Thus, it would be desirable to find additional synthesis routes for these aromatics and related organic chemical compounds that are sustainable, while also providing high-purity end products. Advantageously, the manufacture of such components can be carried out with existing equipment and facilities.

### SUMMARY

In one aspect, the present technology concerns a process for producing a recycled content recycled content organic chemical compound (r-organic chemical compound), the process comprising: (a) processing at least a portion of a recycled content C6 to C10 aromatics (r-C6 to C10 aromatics) stream in an aromatics facility to provide recycled content benzene (r-benzene) stream and a recycled content toluene (r-toluene) stream; and (b) reacting the r-benzene and/or r-toluene in another chemical processing facility to form at least one r-organic chemical compound.

In one aspect, the present technology concerns a process for producing a recycled content organic chemical compound (r-organic chemical compound), the process comprising: (a) forming a recycled content C6 to C10 aromatics (r-C6 to C10 aromatics) stream from a recycled content hydrocarbon (r-HC) stream in at least one conversion unit; (b) processing at least a portion of the r-C6 to C10 aromatics in an aromatics complex to provide recycled content benzene (r-benzene) and/or recycled content toluene (r-toluene); and (c) reacting the r-benzene and/or r-toluene in another chemical processing facility to form at least one r-organic chemical compound.

In one aspect, the present technology concerns a process for producing a recycled content organic chemical compound (r-organic chemical compound), the process comprising: processing at stream comprising recycled content toluene (r-toluene) and/or a stream comprising recycled content benzene (r-benzene) in at least one chemical processing facility to provide at least one r-organic chemical compound, wherein at least a portion of the r-toluene and/or r-benzene is obtained by processing a recycled content C6 to C10 aromatics (r-C6 to C10 aromatics) stream in an aromatics complex and wherein at least a portion of the r-C6 to C10 aromatics is obtained by processing a recycled content hydrocarbon stream in a steam cracking facility and/or in a reformer facility.

In one aspect, the present technology concerns a process for producing a recycled content organic chemical compound (r-organic chemical compound), the process comprising: (a) pyrolyzing waste plastic in a pyrolysis facility to provide a recycled content pyrolysis stream (r-pyrolysis stream); (b) introducing at least a portion of the r-pyrolysis stream into a steam cracking facility to provide a recycled content pyrolysis gasoline (r-pyrolysis gasoline); (c) processing at least a portion of the r-pyrolysis gasoline in an aromatics complex to provide recycled content benzene (r-benzene) and/or toluene (r-toluene); and (d) reacting the r-benzene and/or r-toluene in another chemical processing facility to form at least one r-organic chemical compound.

In one aspect, the present technology concerns a process for producing a recycled content organic chemical compound (r-organic chemical compound), the process comprising: (a) pyrolyzing waste plastic in a pyrolysis facility to provide a recycled content pyrolysis stream (r-pyrolysis stream); (b) processing at least a portion of the r-pyrolysis stream in a reformer unit of a refinery to provide a recycled content reformate (r-reformate) stream; (c) processing at least a portion of the r-reformate stream in an aromatics complex to provide recycled content benzene (r-benzene) and/or toluene (r-toluene); and (d) reacting the r-benzene and/or r-toluene in another chemical processing facility to form at least one r-organic chemical compound.

### BRIEF DESCRIPTION OF THE DRAWINGS

FIG. 1a is a block flow diagram illustrating the main steps of a process for making recycled content aromatics (r-aromatics) and recycled content benzene (r-benzene) and/or recycled content toluene (r-toluene), and optionally, a recycled content organic chemical compound from the r-benzene or r-toluene, wherein the r-aromatics (and r-benzene or r-toluene and r-organic chemical compound) have physical content from one or more source materials;

FIG. 1b is a block flow diagram illustrating the main steps of a process for making r-aromatics (and r-benzene or r-toluene) and optionally, an r-organic chemical compound from the r-benzene or r-toluene, wherein the r-aromatics (and r-benzene or r-toluene and r-organic chemical compound) have credit-based recycled content from one or more source materials;

FIG. 2 is a schematic block flow diagram illustrating the main processes/facilities in a system for providing recycled content organic chemical compounds, including organic chemical compounds derived from r-benzene, r-toluene, and r-xylene, according to various embodiments of the present invention;

FIG. 3 is a schematic block flow diagram illustrating the main steps/zones in an aromatics complex suitable for use in the system illustrated in FIG. 2;

FIG. 4a is a schematic block flow diagram illustrating several possible chemical pathways for producing a variety of benzene-derived recycled content organic chemical compounds (r-organic chemical compounds);

FIG. 4b is another schematic block flow diagram illustrating several possible chemical pathways for producing additional benzene-derived recycled content organic chemical compounds (r-organic chemical compounds);

FIG. 4c is yet another schematic block flow diagram illustrating several possible chemical pathways for producing further benzene-derived recycled content organic chemical compounds (r-organic chemical compounds);

FIG. 4d is still another schematic block flow diagram illustrating several possible chemical pathways for producing even more benzene-derived recycled content organic chemical compounds (r-organic chemical compounds);

FIG. 5a is a schematic block flow diagram illustrating several possible chemical pathways for producing a variety of toluene-derived recycled content organic chemical compounds (r-organic chemical compounds); and

FIG. 5b is another schematic block flow diagram illustrating several possible chemical pathways for producing additional toluene-derived recycled content organic chemical compounds (r-organic chemical compounds).

### DETAILED DESCRIPTION

We have discovered new methods and systems for producing benzene and toluene, as well as organic chemical compounds formed by directly processing these compounds. More specifically, we have discovered a process and system for producing benzene and toluene, where recycled content from waste materials, such as waste plastic, are applied to these aromatics (or their derivatives) in a manner that promotes the recycling of waste plastic and provides benzene and toluene (or other chemical compounds derived from these aromatics) with substantial amounts of recycled content.

In particular, we have discovered new methods and systems for producing benzene and/or toluene and organic chemical compounds formed by directly processing benzene and/or toluene or derivatives thereof. More specifically, we have discovered a process and system for producing benzene and/or toluene where recycled content from waste materials, such as waste plastic, are applied to benzene and/or toluene (or derivatives thereof) in a manner that promotes the recycling of waste plastic and provides benzene and/or toluene (or other organic chemical compounds) with substantial amounts of recycled content.

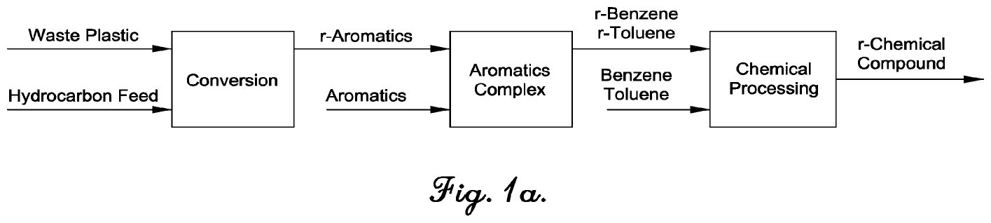

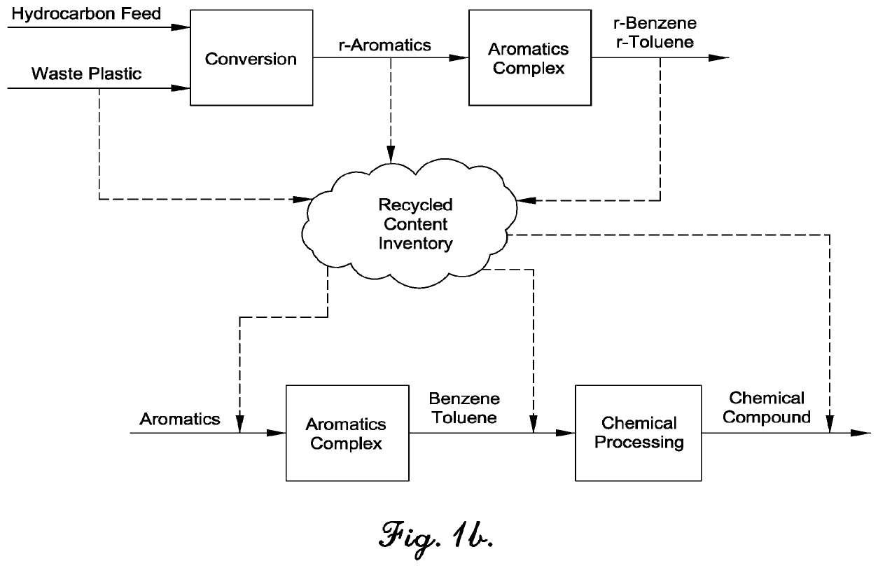

Turning initially to FIGS. 1a and 1b, benzene and toluene are formed by processing a predominantly aromatics stream in an aromatics complex to provide a stream including at least 85, at least 90, at least 92, at least 95, at least 97, or at least 99 weight percent benzene and another with similar amounts of toluene. These streams can undergo one or more additional processing steps to provide at least one organic chemical compound derived from benzene and/or toluene.

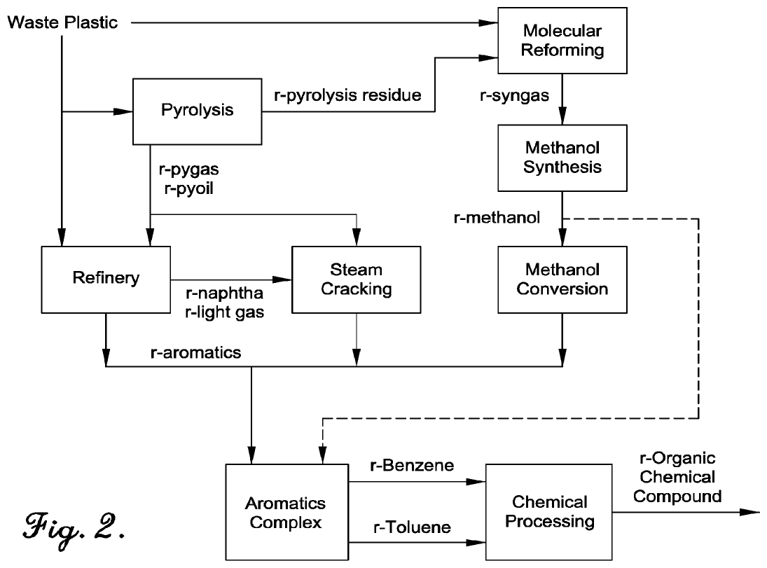

As generally shown in FIGS. 1a and 1b, a stream of waste plastic processed in one or more conversion facilities may provide the aromatics stream, which can be processed to form the benzene and toluene streams. The recycled content in the benzene and toluene streams can be physical and may directly originate from waste plastic or an intermediate hydrocarbon stream formed by processing waste plastic (not shown in FIG. 1 or 2), and/or the recycled content may be credit based and can be applied to a target stream in the aromatics complex and/or chemical processing facilities.

The aromatics (or benzene or toluene or organic chemical compound) streams can have a total recycled content of at least 5, at least 10, at least 15, at least 20, at least 25, at least 30, at least 35, at least 40, at least 45, at least 50, at least 55, or at least 65 percent and/or 100 percent, or less than 99, less than 95, less than 90, less than 85, less than 80, less than 75, or less than 70 percent. Similarly, the r-TPA and/or r-PET or even the r-aromatics stream can have a recycled content of at least 5, at least 10, at least 15, at least 20, at least 25, at least 30, at least 35, at least 40, at least 45, at least 50, at least 55, or at least 65 percent and/or 100 percent, or less than 99, less than 95, less than 90, less than 85, less than 80, less than 75, or less than 70 percent. The recycled content in one or more of these streams can be physical recycled content, credit-based recycled content, or a combination of physical and credit-based recycled content.

Turning initially to FIG. 1a, in one embodiment or in combination with one or more embodiments mentioned herein, at least a portion of the recycled content in the aromatics and/or or benzene or toluene stream (or in the organic chemical compound product stream) can be physical (direct) recycled content. This recycled content may originate from a waste plastic stream. The waste plastic stream is ultimately converted in one or more conversion facilities (e.g., a pyrolysis facility, a refinery, a steam cracking facility, and/or a molecular reforming facility and methanol-to-aromatics facility), which is processed (alone or with a non-recycled content aromatics stream) as described herein to provide an r-benzene stream and an r-toluene streams. These streams can then be further processed (along or in combination with a non-recycled content benzene or toluene stream) to provide one or more recycled content organic chemical compounds.

The amount of physical recycled content in the target product (e.g. composition, r-aromatics or r-benzene or r-toluene or r-organic chemical compound) can be determined by tracing the amount of waste plastic material processed along a chain of chemical pathway(s) and ending with the moiety or portion of the target product attributable to the waste plastic chemical pathway. As used herein, a moiety can be a portion the atoms and their structure of a target product and can also include the entire chemical structure of the target product, and does not necessarily require the inclusion of a functional group. For example, a moiety of toluene can include the aromatic ring, a portion of the aromatic ring, the methyl groups, or the entire toluene molecule. The chemical pathway includes all chemical reactions and other processing steps (e.g., separations) between the starting materials (e.g., waste plastic) and the moiety in the target product attributable to the chemical pathway originating in waste plastics.

For example, the chemical pathway for the r-aromatics can include pyrolysis, optionally refining and/or stream cracking, and/or molecular reforming and methanol synthesis and conversion. The chemical pathway for the r-benzene or r-toluene can further include processing in the aromatics complex, and the chemical pathway for the r-organic chemical compound may include a variety of additional steps, such as, for example, oxidation, polymerization, etc., depending on the specific r-organic chemical compound. A conversion factor may be associated with each step along the chemical pathway. The conversion factors account for the amount of the recycled content diverted or lost at each step along the chemical pathway. For example, the conversion factors can account for the conversion, yield, and/or selectivity of the chemical reactions along the chemical pathway.

The amount of credit-based recycled content in a target product (e.g. compositions, r-aromatics or r-benzene or r-toluene or r-organic chemical compounds) can be determined by calculating the mass weight percent of a target moiety in a target product and attributing a recycle content credit to the target product in any amount up to the mass weight percent of the target moiety in the target product as a maximum. The credit based recycle content that is eligible to be applied to the target product is determined by tracing the waste plastic material along a chain of chemical pathway(s) and ending with the same moiety as target moiety in the target product. Thus, the credit based recycle content can be applied to a variety of different target products having the same moiety even though the products are made by entirely different chemical pathways provided that the credit applied is obtained from waste plastic and the waste plastic ultimately undergoes at least one chemical pathway originating from waste plastic and ending in the target moiety. For example, if a recycle content credit is obtained from waste plastic and booked into a recycle content inventory, and there exists chemical pathways at the facility capable of processing the waste plastic through to a target moiety such as toluene (e.g. a pyrolysis reactor effluent to a crude distillation column to a hydrotreater to a reformer to an aromatics complex that isolates p-xylene), the recycle content credit is then a type eligible to apply to any toluene molecule made by any chemical pathway, including the one existing at the facility and/or to the toluene portion of a pyrolysis gasoline stream composition obtained from a steam cracker and gasoline fractionator. As with physical recycled content, a conversion factor may or may not be associated with each step along the chemical pathway. Additional details on credit-based recycled content are provided below.

The amount of recycled content applied to the r-aromatics (or r-benzene or r-toluene or r-organic chemical compound) can be determined using one of variety of methods for quantifying, tracking, and allocating recycled content among various materials in various processes. One suitable method, known as “mass balance,” quantifies, tracks, and allocates recycled content based on the mass of the recycled content in the process. In certain embodiments, the method of quantifying, tracking, and allocating recycled content is overseen by a certification entity that confirms the accuracy of the method and provides certification for the application of recycled content to the r-aromatics (or r-benzene or r-toluene or r-organic chemical compound).

Turning now to FIG. 1b, one embodiment where the r-organic chemical compound (or r-benzene or r-toluene) includes credit-based recycled content, is provided. Recycled content credits from waste plastic are attributed to one or more streams within the facility. For example, the recycled content credits derived from waste plastics can be attributed to the aromatics stream fed to the aromatics complex, or to any of the products separated and isolated in the aromatics complex, such as to the benzene and/or toluene stream. Alternatively, or in addition, recycled content credits obtained from one or more intermediate streams within the conversion facility and/or aromatics complex can also be attributed to one or more products, such as benzene and/or toluene, within the facility, depending on the specific configuration of the system. Further, recycled content credits from one or more of these streams may also be attributed to the organic chemical compound stream, as shown in FIG. 1b.

As such, the waste plastic stream, or the r-aromatics stream and or r-benzene and/or r-toluene streams (and any recycled content intermediate streams not shown in FIG. 1b) not made at the facility or purchased or acquired, can each act as a “source material” of recycled content credits. The aromatics fed to the aromatics complex, the benzene and/or toluene product or any other products separated and/or isolated from the aromatics complex, the benzene and/or toluene transferred (including sales) or fed to the chemical processing facility, any intermediate streams not shown, and even the organic chemical compound, can each act as a target product to which the recycled content credits are attributed. In one embodiment or in combination with any embodiment mentioned herein, the source material has physical recycled content and the target product has less than 100 percent physical recycled content. For example, the source material can have at least 10, at least 25, at least 50, at least 75, at least 90, at least 99, or 100 percent physical recycled content and/or the target product can have less than 100, less than 99, less than 90, less than 75, less than 50, less than 25, less than 10, less than 1 percent, or no physical recycled content.

The ability to attribute recycled content credits from a source material to a target product removes the co-location requirement between the facility making the source material (with physical recycled content) and the facility making the aromatics or products receiving recycle content value (e.g. benzene and/or toluene or organic chemical compound). This allows a chemical recycling facility/site in one location to process waste material into one or more recycled content source materials and then apply recycled content credits from those source materials to one or more target products being processed in existing commercial facilities located remotely from the chemical recycling facility/site, optionally within the same Family of Entities, or to associate a recycle content value with a product that is transferred to another facility, optionally owned by a different entity that can deposit the recycle content credit into its recycle content inventory one the product is receiving, purchased, or otherwise transferred. Further, the use of recycled content credits allows different entities to produce the source material and the aromatics (or benzene or toluene or organic chemical compound). This allows efficient use of existing commercial assets to produce the aromatics (or benzene or toluene or organic chemical compound). In one or more embodiments, the source material is made at a facility/site that is at least 0.1, at least 0.5, at least 1, at least 5, at least 10, at least 50, at least 100, at least 500, or at least 1000 miles from the facility/site where the target product is used to make the aromatics (or benzene or toluene or organic chemical compound).

The attributing of recycled content credits from the source material (e.g., the r-aromatics from the conversion facility) to the target product (e.g., an aromatics stream fed to an aromatics complex) can be accomplished by transferring recycled content credits directly from the source material to the target product. Alternatively, as shown in FIG. 1b, recycled content credits can be applied from any of the waste plastic, r-aromatics, and r-benzene or r-toluene (when present) to the aromatics, benzene, toluene, or organic chemical compound via a recycled content inventory.

When a recycled content inventory is used, recycled content credits from the source material having physical recycled content (e.g., the waste plastic, the r-aromatics, and optionally the r-benzene and/or r-toluene shown in FIG. 1b) are booked into the recycled content inventory. The recycled content inventory can also contain recycled content credits from other sources and from other time periods. In one embodiment, recycled content credits in the recycled content inventory correspond to a moiety, and the recycle content credit is applied or assigned to the same a target products containing a target moiety, and the target moiety is either (i) not chemically traceable through chemical pathways used to for generating the recycle content credit or (ii) is chemically traceable through chemical pathways used for generating the recycle content credit. Chemical traceability is achieved when atoms from a source material such as waste plastic can be theoretically traced to one or more atoms in the target moiety of a target product through each chemical pathway to obtain that atom(s) in the target moiety.

In some embodiments, there may be a periodic (e.g., annual or semi-annual) reconciliation between waste plastic credits deposited in the recycled content inventory and the mass of waste plastic processed. Such reconciliations may be performed by an appropriate entity at an interval consistent with rules of the certification system in which the producer is participating.

In one embodiment, once recycled content credits have been attributed to the target product (e.g., the aromatics stream, the benzene or toluene stream, or any intermediate stream not shown), the amount of the credit-based recycled content allocated to the organic chemical compound (e.g., TPA, PET, or other organic chemical compound) is calculated by the mass proportion of atoms in the target product that are chemically traceable to the source material. In another embodiment, a conversion factor can be associated with each step along the chemical pathway of the credit-based recycled content. The conversion factors account for the amount of the recycled content diverted or lost at each step along the chemical pathway. For example, the conversion factors can account for the conversion, yield, and/or selectivity of the chemical reactions along the chemical pathway. However, if desired, the amount of recycle content applied to a target product can be more than the mass proportion of the target moiety chemically traceable to the waste plastic source material. The target product can receive up to 100% recycle content even though the mass proportion of atoms in the target moiety that is chemically traceable to a recycle source material, such as mixed plastic waste stream, is less than 100%. For example, if the target moiety in a product represents only 30 wt. % of all atoms in a target product that are chemically traceable to a mixed plastic waste stream, the target product can nevertheless receive more than 30% recycle content value, up to 100% if desired. While such application would violate chemical traceability for the full value of the amount of recycle content in a target product back to a waste plastic source, the particular amount of recycle content value applied to a target product will depend on the rules of a certification system that the producer participates in.

As with the physical recycled content, the amount of credit-based recycled content applied to the r-aromatics (or r-benzene or r-toluene or r-organic chemical compound) can be determined using one of variety of methods, such as mass balance, for quantifying, tracing, and allocating recycled content among various products in various processes. In certain embodiments the method of quantifying, tracing, and allocating recycled content is overseen by a certification entity that confirms the accuracy of the method and provides certification for the application of recycled content to the r-aromatics (or r-benzene or r-toluene or r-organic chemical compound).

The r-aromatics (or r-benzene or r-toluene or r-organic chemical compound) can have 25 to 90, 40 to 80, or 55 to 65 percent credit-based recycled content and less than 50, less than 25, less than 10, less than 5, or less than 1 percent physical recycled content. In certain embodiments, the r-aromatics (or r-benzene or r-toluene or r-organic chemical compound) can have at least 10, at least 25, at least 50, or at least 65 percent and/or not more than 90, not more than 80, or not more than 75 percent credit-based recycled content from one or more of the r-aromatics and/or r-benzene and/or r-toluene, individually.

In one or more embodiments, the recycled content of the r-aromatics (or r-benzene or r-toluene or r-organic chemical compound) can include both physical recycled content and credit-based recycled content. For example, the r-aromatics (or r-benzene or r-toluene or r-organic chemical compound) can have at least 10, at least 20, at least 30, at least 40, or at least 50 percent physical recycled content and at least 10, at least 20, at least 30, at least 40, or at least 50 percent credit-based recycled content. As used herein, the term “total recycled content” refers to the cumulative amount of physical recycled content and credit-based recycled content from all sources.

Turning now to FIG. 2, a process and facility for use in forming a recycled content organic chemical compound (r-organic chemical compound) is provided. As used herein, the term “organic chemical compound,” refers to a chemical compound that includes carbon and hydrogen atoms, but also includes oxygen and/or nitrogen atoms. An organic chemical compound can include at least 75, at least 80, at least 85, at least 90, at least 95, or at least 99 atom percent of carbon and hydrogen atoms combined, with the balance being nitrogen and oxygen.

Specifically, the system shown in FIG. 2 illustrates several types of waste plastic conversion facilities (e.g., a pyrolysis facility, a refinery, a steam cracking facility, a molecular reforming facility and related methanol-to-aromatics conversion facilities, and an optional PET cracking facility) for processing a stream of waste plastic (and/or one or more streams derived from waste plastic) to provide streams of recycled content aromatics (r-aromatics). Additionally, although not shown in FIG. 2, each of these conversion facilities may also process a conventional stream of hydrocarbon-containing materials along with the waste plastic and/or stream derived from waste plastic. For example, the refinery may also process crude oil, the steam cracking facility may also process a hydrocarbon stream (e.g., light gas and/or naphtha), and the molecular reforming facility may also process at least one hydrocarbon-containing stream (e.g., coal, petroleum, etc.) Further, the aromatics complex may also receive and process another aromatics-containing stream not from one or more of the conversion facilities. These additional feed streams may or may not include recycled content.

As shown in FIG. 2, the r-aromatics stream or stream from one or more of the conversion facilities may then be further processed in an aromatics complex to provide a stream of recycled content benzene (r-benzene) and/or recycled content toluene (r-toluene), which can then be further processed to form recycled content organic chemical compounds (r-organic chemical compounds) derived from benzene and/or toluene.

The system shown in FIG. 2 can include or be a chemical recycling facility. Chemical recycling facilities are not the same as mechanical recycling facilities. As used herein, the terms “mechanical recycling” and “physical recycling” refer to a recycling process that includes a step of melting waste plastic and forming the molten plastic into a new intermediate product (e.g., pellets or sheets) and/or a new end product (e.g., bottles). Generally, mechanical recycling does not substantially change the chemical structure of the plastic being recycled. The chemical recycling facilities described herein may be configured to receive and process waste streams from and/or that are not typically processable by a mechanical recycling facility.

In one embodiment or in combination with any embodiments mentioned herein, at least two, at least three, at least four, at least five, at least six, or all of the pyrolysis facility, the refinery, the steam cracking facility, the molecular reforming facility, the methanol-to-aromatics conversion facility, the aromatics complex, and the chemical processing facility may be co-located. As used herein, the term “co-located” refers to the characteristic of at least two objects being situated on a common physical site, and/or within 5, within 3, within 1, within 0.75, within 0.5, or within 0.25 miles of each other, measured as a straight-line distance between two designated points. When two or more facilities are co-located, the facilities may be integrated in one or more ways. Examples of integration include, but are not limited to, heat integration, utility integration, waste-water integration, mass flow integration via conduits, office space, cafeterias, integration of plant management, IT department, maintenance department, and sharing of common equipment and parts, such as seals, gaskets, and the like.

Additionally, one or more, two or more, three or more, four or more, five or more six, or all, of the pyrolysis facility, the refinery, the steam cracking facility, the molecular reforming facility, the methanol-to-aromatics conversion facility, the aromatics complex, and the chemical processing facility may be commercial-scale facilities. For example, in one embodiment or in combination with any embodiments mentioned herein, one or more of these facilities/steps can accept one or more feed streams at a combined average annual feed rate of at least 500, at least 1000, at least 1500, at least 2000, at least 5000, at least 10,000, at least 50,000, or at least 100,000 pounds per hour, averaged over one year. Further, one or more of the facilities can produce at least one recycled content product streams at an average annual rate of at least 500, or at least 1000, at least 1500, at least 2000, at least 2500, at least 5000, at least 10,000, at least 50,000, or at least 75,000 pounds per hour, averaged over one year. When more than one r-product stream is produced, these rates can apply to the combined rate of all r-products.

One or more, two or more, three or more, four or more, five or more, six, or all, of the pyrolysis facility, the refinery, the steam cracking facility, the molecular reforming facility, the methanol-to-aromatics conversion facility, the aromatics complex, and the chemical processing facility can be operated in a continuous manner. For example, each of the steps or processes within each of the facilities and/or the process amongst the facilities may be operated continuously and may not include batch or semi-batch operation. In one embodiment or in combination with any embodiments mentioned herein, at least a portion of one or more of the facilities may be operated in a batch or semi-batch manner, but the operation amongst the facilities may be continuous overall.

As shown in FIG. 2, waste plastic may be introduced into one or more conversion facilities for processing the waste plastic (or hydrocarbon streams derived from waste plastic) to form recycled content products. Examples of conversion facilities shown in FIG. 2 include a pyrolysis facility, a refinery, a steam cracking facility, and a molecular reforming facility (with methanol-to-aromatics conversion facility). In some embodiments (not shown in FIG. 2), the conversion facility may include a PET cracking facility. A single chemical recycling complex may include one or more of these facilities or several conversion facilities may be in separate locations (i.e., not co-located). As shown in FIG. 2, these facilities may work independently or in combination to provide a stream of recycled content aromatics (r-aromatics), which can then be processed in an aromatics complex to form streams of r-benzene and/or r-toluene that can be further reacted to form a variety of plasticizers, intermediate chemicals, and even polymers. The basic operation of these facilities will now be discussed in further detail below.

In one embodiment or in combination with any embodiment mentioned here, a stream of mixed waste plastic may be passed through a plastics processing facility (not shown) and the processed waste plastic may be introduced into one or more of the conversion units. The plastics processing facility, when present, can separate mixed plastic into PET-enriched and a polyolefin (PO)-enriched streams and these separated streams can be introduced into separate conversion facilities. Additionally, or in the alternative, the plastics processing facility may also reduce the size of the incoming plastic via a crushing, flaking, pelletizing, grinding, granulating, and/or pulverizing step and/or the waste plastic may be melted to form a molten plastic or combined with a liquid to form liquified plastic or a slurry. One or more cleaning or separation steps may also be present to remove dirt, food, sand, glass, aluminum, lignocellulosic materials such as paper and cardboard, from the incoming waste stream.

Referring initially to the pyrolysis facility, waste plastic (and, in some cases, predominantly PO-containing waste plastic) may be introduced into the pyrolysis facility, wherein it can be pyrolyzed to form at least one recycled content pyrolysis effluent (r-pyrolysis effluent) stream. Any suitable pyrolysis facility/step can be used and it may include, for example, at least one pyrolysis reactor for chemically and/or thermally decomposing the waste plastic. Although pyrolysis is generally carried out in a reaction environment that is substantially free of molecular oxygen, pyrolysis processes may be further defined by other parameters such as the pyrolysis reaction temperature within the reactor, the residence time in the pyrolysis reactor, the reactor type, the pressure within the pyrolysis reactor, and the presence or absence of pyrolysis catalysts.

The feed to the pyrolysis reactor can comprise, consists essentially of, or consists of waste plastic, and the feed stream can have a number average molecular weight (Mn) of at least 3000, at least 4000, at least 5000, or at least 6000 g/mole. If the feed to the pyrolysis reactor contains a mixture of components, the Mn of the pyrolysis feed is the average Mn of all feed components, based on the weight of the individual feed components. The waste plastic in the feed to the pyrolysis reactor can include post-consumer waste plastic, post-industrial waste plastic, or combinations thereof. In certain embodiments, the feed to the pyrolysis reactor comprises less than 5, less than 2, less than 1, less than 0.5, or about 0.0 weight percent coal and/or biomass (e.g., lignocellulosic waste, switchgrass, fats and oils derived from animals, fats and oils derived from plants, etc.). The feed to the pyrolysis reaction can also comprise less than 5, less than 2, less than 1, or less than 0.5, or about 0.0 weight percent of a co-feed stream, including steam and/or sulfur-containing co-feed streams. In other cases, steam fed to the pyrolysis reactor can be present in amounts of up to 50 weight percent.

The pyrolysis reaction can involve heating and converting the waste plastic feedstock in an atmosphere that is substantially free of molecular oxygen or in an atmosphere that contains less molecular oxygen relative to ambient air. For example, the atmosphere within the pyrolysis reactor may comprise not more than 5, not more than 4, not more than 3, not more than 2, not more than 1, or not more than 0.5 weight percent of molecular oxygen. The pyrolysis reaction in the reactor can be thermal pyrolysis, which is carried out in the absence of a catalyst, or catalytic pyrolysis, which is carried out in the presence of a catalyst. When a catalyst is used, the catalyst can be homogenous or heterogeneous and may include, for example, oxides, certain types of zeolites and other mesostructured catalysts.

The pyrolysis reactor may have any suitable design and can comprise a film reactor, a screw extruder, a tubular reactor, a stirred tank reactor, a riser reactor, a fixed bed reactor, a fluidized bed reactor, a rotary kiln, a vacuum reactor, a microwave reactor, or an autoclave. The reactor may also utilize a feed gas and/or lift gas for facilitating the introduction of the feed into the pyrolysis reactor. The feed gas and/or lift gas can comprise nitrogen and can comprise less than 5, less than 2, less than 1, or less than 0.5, or about 0.0 weight percent of steam and/or sulfur-containing compounds. The feed and/or lift can also include light hydrocarbons, such a methane, or hydrogen, and these gases may be used alone or in combination with steam.

After exiting the reactor, a stream of recycled content pyrolysis effluent (r-pyrolysis effluent) can be separated to form recycled content pyrolysis streams, including recycled content pyrolysis residue (r-pyrolysis residue) and recycled content pyrolysis vapor (r-pyrolysis vapor), or the r-pyrolysis vapor may be further separated to provide streams of recycled content pyrolysis gas (r-pygas) and recycled content pyrolysis oil (r-pyoil). In some cases, the second separation step may be omitted so that the stream of r-pyrolysis vapor is removed from the facility and introduced into a downstream processing facility.

When withdrawn as a separate product stream, the r-pyoil can include predominantly C5 to C22 hydrocarbon components, or it can include at least 55, at least 60, at least 65, at least 70, at least 75, or at least 80 weight percent of C5 to C22 hydrocarbon components, while the r-pygas can include predominantly C2 to C4 hydrocarbon components, or at least 30, at least 40, at least 45, at least 55, at least 60, at least 65, at least 70, at least 75, or at least 80 weight percent C2 to C4 hydrocarbon components. In some cases, the C2 to C4 components in the r-pygas can include at least 50, at least 55, at least 60, at least 65, at least 70, or at least 75 weight percent of alkanes and/or at least 40, at least 45, at least 50, at least 55, at least 60, at least 65, at least 70, or at least 75 weight percent of olefins, based on the amount of C2 to C4 hydrocarbon components in the stream. The r-pyrolysis residue stream may include at least 55, at least 65, at least 75, at least 85, or at least 90 weight precent of C20 and heavier hydrocarbons (e.g., pyrolysis waxes), as well as carbon-containing components that are solid at 200° C. and 1 atmosphere, absolute (e.g., pyrolysis char).

The r-pyoil may also comprise one or more of the following (i) through (v): (i) less than 500 ppm, less than 450 ppm, less than 350 ppm, less than 250 ppm, less than 100 ppm, less than 75 ppm, less than 50 ppm, less than 25 ppm, or less than 10 ppm sulfur; (ii) less than 300 ppm, less than 150 ppm, less than 100 ppm, less than 50 ppm, less than 25 ppm, less than 10 ppm, or less than 5 ppm chlorine; (iii) less than 500 ppm, less than 250 ppm, less than 100 ppm, less than 75 ppm, less than 50 ppm, less than 30 ppm, or less than 20 ppm water; (iv) less than 500 ppb, less than 250 ppb, less than 100 ppb, less than 50 ppb, less than 25 ppb, less than 10 ppb, less than 5 ppb, or less than 2 ppb arsenic; and/or (v) less than 1500 ppm, less than 1000 ppm, less than 500 ppm, less than 250 ppm, less than 100 ppm, less than 75 ppm, less than 50 ppm, less than 30 ppm, or less than 20 ppm nitrogen.

As shown in FIG. 2, at least a portion of the r-pyrolysis residue may be introduced into the molecular reforming facility alone or in combination with a stream of waste plastic and/or other feed stream (not shown), which may or may not include recycled content. Examples of the other feed stream introduced into the molecular reforming facility can include, but are not limited to, coal, petroleum coke, lignocellulosic materials, liquid hydrocarbons, natural gas, organic hydrocarbons, and mixtures thereof. When introduced into the molecular reforming facility, the waste plastic can be in the form of a solid powder and/or in the form of a slurry with water or other liquid.

As used herein, the term “molecular reforming” refers to conversion of a carbon-containing feed into syngas (CO, CO2, and H2). Molecular reforming encompasses both steam reforming and partial oxidation (POX) gasification. As used herein, the term “steam reforming” refers to the conversion of a carbon-containing feed into syngas (i.e., a gas stream comprising at least 90, at least 95, at least 97, or at least 99 weight percent carbon monoxide, hydrogen, and carbon dioxide) via reaction with water. The steam reforming can include, for example, steam-methane reforming in which the carbon-containing feed includes a methane-containing stream, such as natural gas. As used herein, the term “partial oxidation (POX) gasification” or “POX gasification” refers to high temperature conversion of a carbon-containing feed into syngas, where the conversion is carried out in the presence of a less-than-stoichiometric amount of oxygen. The carbon-containing feed to POX gasification can include solids, liquids, and/or gases and may, in some cases, include waste plastic. When one or more feed streams to the molecular reforming facility include waste plastic or recycled content derived from waste plastic (or another source), the syngas produced is recycled content syngas (r-syngas). The r-syngas may further include non-recycled content when a portion of the feed does not include or is not derived from waste plastic.

As shown in FIG. 2, at least a portion of the r-syngas formed in the molecular reforming facility may be introduced into a methanol-to-aromatics conversion facility. The methanol-to-aromatics (MTA) conversion facility includes a methanol synthesis step for synthesizing methanol from syngas (or synthesizing recycled content methanol, r-methanol, from r-syngas) and a methanol conversion step for converting r-methanol to a stream of recycled content aromatics (r-aromatics). In some cases, the MTA conversion facility may first react a stream of methanol (or r-methanol) at temperatures of around 400 to 600° C., or 450 to 500° C., over a selective catalyst (e.g., ZSM) to form a mixture of aromatics, olefins, and alkanes. Some of the heavier alkanes and/or olefins can be recycled, along with at least a portion of the benzene and/or toluene, to increase conversion, while the lighter alkanes may be further reacted at a higher temperature of 500 to 600° C. to form additional aromatics (r-aromatics), which can be further processed (e.g., separated) to provide a recycled content aromatics (r-aromatics) stream as shown in FIG. 2. The resulting r-aromatics stream exiting the methanol-to-aromatics conversion facility can include recycled content benzene, toluene, and xylenes (r-BTX), and may include, for example, at least 35, at least 40, at least 45, or at least 50 weight percent and/or not more than 95, not more than 85, not more than 75, not more than 70, not more than 65, or not more than 60 weight percent of these components.

Turning back to FIG. 2, when the chemical recycling facility includes a refinery, at least a portion of the r-pyoil and/or r-pygas (or r-pyrolysis vapor if not separated in the pyrolysis facility) may be introduced into one or more locations of the refinery to undergo at least one processing step to provide one or more recycled content hydrocarbon streams. Examples of recycled content hydrocarbon streams produced by the refinery can include, but are not limited to, recycled content light gas (r-light gas), recycled content naphtha (r-naphtha), and recycled content aromatics (r-aromatics). Additionally, a stream of waste plastic, typically liquified waste plastic, can also be processed in at least one unit within the refinery to provide these recycled content streams.

The processing steps utilized in the refinery can include separation or distillation, cracking, and reforming, along with other processing steps for removing sulfur, nitrogen, and other impurities. In some cases, the r-pyoil and/or r-pyrolysis vapor can be introduced into an atmospheric distillation column (ADU) and can be separated along with a crude oil feedstock to form several recycled content hydrocarbon fractions. Lighter fractions, such as r-light gas, can be further separated to remove impurities, while heavier fractions, such as r-gas oils, can be introduced into gas oil crackers and thermally and/or catalytically cracked to provide recycled content cracked light gas (r-cracked light gas) and recycled content cracked naphtha (r-cracked naphtha). At least a portion of the r-cracked naphtha, along with the r-naphtha removed from the ADU, can be introduced into a reformer unit, wherein it can be converted to a recycled content reformate (r-reformate) stream. The r-reformate stream may comprise predominantly C6 to C10 aromatics and at least a portion of this stream can be withdrawn from the refinery as the r-aromatics stream shown in FIG. 2.

When the chemical recycling facility includes a steam cracking facility, at least a portion of the r-light gas and/or r-naphtha from the refinery and/or at least a portion of the r-pygas and/or r-pyoil from the pyrolysis facility can be introduced into the steam cracking facility. In some cases, gas-phase streams (e.g., r-pygas and/or r-light gas, optionally with another predominantly C2 to C4 gas stream, with or without recycled content) can be introduced into the inlet of a steam cracker furnace in the steam cracking facility, while, in other cases, these streams may be introduced into one or more locations downstream of the furnace. When one or more liquid-phase streams (e.g., r-pyoil and/or r-naphtha, optionally with another predominantly C5 to C22 liquid stream, with or without recycled content) are introduced into the steam cracking facility, these streams can be fed to the inlet of the steam cracking furnace.

In the steam cracking furnace, the hydrocarbon feed stream, which can include one or more of r-pygas, r-pyoil, r-light gas, and r-naphtha, can be thermally cracked in the presence of steam to form a predominantly recycled content olefin-containing (r-olefin-containing) stream and a stream of recycled content pyrolysis gasoline (r-pyrolysis gasoline). The r-olefin-containing stream may be compressed and further processed in a separation zone of the steam cracking facility to provide one or more recycled content olefin (r-olefin) products (e.g., r-ethylene and/or r-propylene), while a recycled content pyrolysis gasoline (r-pyrolysis gasoline), which comprises predominantly C6 to C10 aromatics, can be withdrawn from the steam cracking facility as the r-aromatics stream shown in FIG. 2.

The r-aromatics stream or streams withdrawn from each of the refinery, the steam cracking facility, the MTA conversion facility (or two or more, or all of these facilities combined) can have one or more of the following properties (i) through (viii): (i) the stream(s) can comprise predominantly C6 to C10 (or C6 to C9) aromatics, or it can include at least 25, at least 35, at least 45, at least 55, at least 60, at least 65, at least 70, at least 75, at least 80, at least 85, or at least 90 weight percent of C6 to C10 (or C6 to C9) aromatic components; (ii), the stream(s) can comprise less than 75, less than 65, less than 55, less than 45, less than 35, less than 25, less than 15, or less than 10 weight percent of non-aromatic components; (iii) the streams can comprise at least 1, at least 2, at least 3, at least 5, or at least 10 and/or not more than 30, not more than 25, not more than 20, not more than 15, not more than 10, or not more than 7 weight percent of benzene, which may include recycled content benzene (r-benzene) and/or non-recycled content benzene; (iv) the stream(s) can comprise at least 5, at least 10, at least 15, or at least 20 and/or not more than 40, not more than 35, not more than 30, not more than 25, or not more than 20 weight percent of toluene, which can include both recycled content toluene (r-toluene) and/or non-recycled content toluene; (v) the stream(s) can comprise at least 2, at least 5, at least 10, at least 15, at least 20, or at least 25 weight percent and/or not more than 75, not more than 70, not more than 65, not more than 60, not more than 55, not more than 50, not more than 45, not more than 40, not more than 35, not more than 30, or not more than 25 weight percent of one or more of C8 aromatics (or recycled content C8 aromatics, r-C8 aromatics), C9 aromatics (or recycled content C9 aromatics, r-C9 aromatics), and C10 aromatics (or recycled content C10 aromatics, r-C10 aromatics, individually or in combination; (vi) the stream(s) can comprise at least 5, at least 10, or at least 15 and/or not more than 50, not more than 45, or not more than 40 weight percent of mixed xylenes, including recycled and non-recycled content xylenes; (vii) the stream(s) may comprise not more than 15, not more than 10, not more than 5, not more than 2, or not more than 1 weight percent of C5 and lighter components and/or C11 and heavier components; and (viii) the stream(s) can comprise a total amount of C6 to C10 (or C9 to C10) hydrocarbon components of at least 55, at least 60, at least 65, at least 70, at least 75, at least 80, at least 85, or at least 90 weight percent, based on the total weight of the stream

Examples of C8 aromatics include, but are not limited to, mixed xylenes such as ortho-xylene, paraxylene, and meta-xylene, as well as ethylbenzene, and styrene, while C9 aromatics can include, for example, isopropyl benzene, propyl benzene, isomers of methyl ethyl benzene, isomers of methyl styrene, and isomers of trimethyl benzene. Examples of C10 aromatics can include, but are not limited to, isomers of butyl benzene, isomers of diethyl benzene, and isomers of dimethyl ethyl benzene. One or more of these components, when present in the aromatics stream, may include recycled content and/or may include non-recycled content.

In one embodiment or in combination with any embodiment mentioned herein, the r-aromatics stream may comprise 20 to 80, or 25 to 75, or 30 to 60 weight percent benzene and/or 0.5 to 40, or 1 to 35, or 2 to 30 weight percent toluene, and/or 0.05 to 30, or 0.10 to 25, or 0.20 to 20 weight percent of C8 aromatics, based on the total weight of aromatics in the r-aromatics stream.

As shown in FIG. 2, at least a portion of one or more or all of the r-aromatics streams from the conversion facilities (e.g., refinery, steam cracking facility, and/or MTA conversion facility) can be introduced into an aromatics complex, wherein it can be processed to form at least one recycled content aromatics product stream. The r-aromatics stream introduced into the aromatics complex can undergo several processing steps, including, but not limited to, separation (e.g., distillation, extraction, crystallization, adsorption, and combinations thereof), isomerization, alkylation, and transalkylation/disproportionation. The resulting recycled content aromatic products withdrawn from the aromatics complex can include, for example, recycled content paraxylene (r-paraxylene), recycled content metaxylene (r-metaxylene), and recycled content orthoxylene (r-orthoxylene), as well as streams comprising predominantly recycled content benzene (r-benzene), recycled content toluene (r-toluene), and even recycled content C9 and heavier aromatics (r-C9+). In some cases, each of these streams can include at least 55, at least 60, at least 65, at least 70, at least 75, at least 80, at least 85, at least 90, at least 92, or at least 95 weight percent of the named component, based on the total weight of the stream.

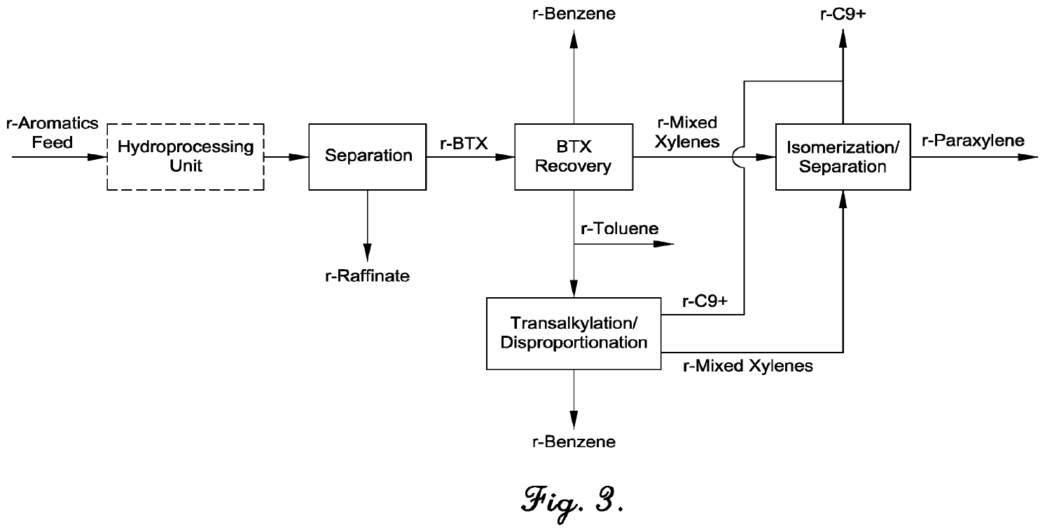

Turning now to FIG. 3, a schematic diagram of the main steps/zones of an aromatics complex as shown in FIG. 2 is provided. As shown in FIG. 3, at least a portion of an r-aromatics stream from one of the conversion facilities may be introduced into an initial separation step of the aromatics complex. In some cases, the r-aromatics stream may optionally be subjected to hydroprocessing prior to the initial separation step, and the hydroprocessed stream may be introduced into the first step of the aromatics complex.

In one embodiment or in combination with any embodiments mentioned herein, the initial separation zone may remove at least 50, at least 60, at least 75, at least 80, or at least 90 weight percent of the total amount of aromatics introduced into the separation zone, resulting in an aromatics-enriched predominantly benzene, toluene, and xylene (BTX) stream and an aromatics-depleted raffinate stream. The BTX stream may comprise at least at least 55, at least 65, at least 75, at least 85, or at least 90 weight percent C6 to C9 aromatics, while the raffinate stream can comprise less than 50, less than 40, less than 30, less than 20, or less than 10 weight percent C6 to C9 aromatics. When one or more of the feed streams to the initial separation zone comprise recycled content, the BTX stream can be a recycled content BTX (r-BTX) stream, and the raffinate stream may be a recycled content raffinate (r-raffinate) stream.

In addition to BTX, the r-BTX stream may include other aromatic and non-aromatic components. For example, the r-BTX (or BTX) stream may include at least 1, at least 2, at least 5, or at least 10 weight percent and/or not more than 25, not more than 20, not more than 15, or not more than 10 weight percent of C9 and heavier (or C10 and heavier) components. Such components can include C9 and heavier (or C10 and heavier) aromatic components as well as non-aromatic C9 and heavier (or C10 and heavier) components.

The initial separation step shown in FIG. 3 for removing BTX from the incoming streams may be performed using any suitable type of separation, including extraction, distillation, and extractive distillation. When the separation step includes extraction or extractive distillation, it may utilize at least one solvent selected from the group consisting of sulfolane, furfural, tetraethylene glycol, dimethylsulfoxide, N,N-dimethylformamide, and N-methyl-2-pyrrolidone. Upon separation, a recycled content raffinate (r-raffinate) stream depleted in aromatics can be withdrawn from the separation step/zone. The r-raffinate stream comprises predominantly C5 to C12 components and may include not more than 20, not more than 15, not more than 10, or not more than 5 weight percent of C6 to C9 aromatics (e.g., benzene, toluene, and xylenes).

Additionally, as shown in FIG. 3, a stream concentrated in recycled content benzene, toluene, and xylenes (r-BTX) can also be withdrawn from the initial separation step. This r-BTX stream comprises predominantly BTX and may include at least 60, at least 70, at least 80, at least 85, at least 90, or at least 95 BTX, including both recycled content BTX (r-BTX) and non-recycled content BTX, as applicable. This stream may also include at least 5, at least 10, at least 15 and/or not more than 30, not more than 25, not more than 20, and/or not more than 10 weight percent of lighter and/or heavier components, such as styrene. The r-BTX stream can be introduced into a downstream BTX recovery zone, which utilizes one or more separation steps to provide streams concentrated in recycled content benzene (r-benzene), recycled content mixed xylenes (r-mixed xylenes), and recycled content toluene (r-toluene). Such separations can be performed according to any suitable method, including, for example, with one or more distillation columns or other separation equipment or steps.

As discussed previously, this r-BTX stream may include other C8 aromatics (such as ethylbenzene), as well as C9 and heavier (or C10 and heavier) components in addition to the benzene, toluene, and mixed xylenes. Components other than BTX in the r-BTX stream may be present in an amount of at least 1, at least 2, at least 5, or at least 10 weight percent and/or not more than 25, not more than 20, not more than 15, or not more than 10 weight percent.

As shown in FIG. 3, the r-benzene formed in BTX recovery step can be removed as a product stream from aromatics complex, while the r-mixed xylenes can be introduced into a second separation step for separating out recycled content ortho-xylene (r-oX), recycled content meta-xylene (r-mX), and/or recycled content paraxylene (r-pX) from the other components in the stream. In addition to comprising at least 25, at least 30, at least 35, at least 40, or at least 45 weight percent and/or not more than 70, not more than 65, not more than 60, or not more than 55 weight percent mixed xylenes, this stream of r-mixed xylenes may also include other C8 aromatics (such as ethylbenzene), as well as C9 and heavier (or C10 and heavier) aromatic and non-aromatic components. Such components (which may include recycled content or non-recycled content components) can be present in the r-BTX stream in an amount of at least 1, at least 2, at least 5, or at least 10 weight percent and/or not more than 35, not more than 30, not more than 25, not more than 20, not more than 15, not more than 10, or not more than 5 weight percent.

This second separation step can utilize one or more of distillation, extraction, crystallization, and adsorption to provide recycle content aromatics streams. For example, as shown in FIG. 3, the separation step can provide at least one of a recycled content paraxylene (r-paraxylene) stream, a recycled content metaxylene (r-metaxylene) stream, and a recycled content orthoxylene (r-orthoxylene) stream. Each of these streams may include both recycled and non-recycled content and can individually include at least 75, at least 80, at least 85, at least 90, at least 95, or at least 97 weight percent of paraxylene (r-pX and pX), metaxylene (r-mX and mX), or orthoxylene (r-oX and oX), respectively. Additionally, at least a portion of the oX (or r-oX) and/or mX (or r-mX) can be subjected to isomerization to provide additional pX (or r-pX). After the isomerization, additional separation steps may be performed to provide individual streams of oX (or r-oX), mX (or r-mX), and pX (or r-pX).

As shown in FIG. 3, a stream of recycled content C9 and heavier components (r-C9+ components) may also be withdrawn from the second separation step and all or a portion may be introduced into a transalkylation/disproportionation step along with a stream of r-toluene withdrawn from the BTX recovery step/zone. In the transalkylation/disproportionation step/zone, at least a portion of the toluene (or r-toluene) can be reacted in the presence of a regenerable fixed bed silica-alumina catalyst to provide mixed xylenes (or r-mixed xylenes) and benzene (or r-benzene). Alternatively, or in addition, at least a portion of the r-toluene can be reacted with methanol (and, optionally, r-methanol) to provide recycled content paraxylene (r-paraxylene), which may be further processed as described herein. In some cases, this reaction may be performed within the aromatics complex over an acidic catalyst, preferably on a shape-selective molecular sieve catalyst such as ZSM-5, and the resulting r-paraxylene may be combined with other paraxylene (or r-paraxylene) recovered in the aromatics complex, as shown in FIG. 3. As also shown in FIG. 3, the benzene (or r-benzene) can be recovered as a product, while the r-mixed xylenes can be introduced into the second separation step/zone for further separation into a r-paraxylene stream, an r-orthoxylene stream, and a r-metaxylene stream.

In one embodiment or in combination with any embodiment mentioned herein, a the r-raffinate stream can be withdrawn from the initial separation zone of the aromatics complex and can be returned to one or more of the conversion facilities for processing to form additional r-aromatics. The r-raffinate can comprise predominantly C5 to C8 hydrocarbon components but may include less than 20, less than 15, less than 10, or less than 5 weight percent of aromatics, and may be returned to the reformer unit in the refinery and/or into the steam cracker furnace of the steam cracking facility to be further processed to form additional streams including recycled content aromatics (r-aromatics).

In one embodiment or in combination with any embodiment mentioned herein, the chemical recycling facility may include a PET cracking facility for thermally cracking waste plastic comprising predominantly PET. The feed to the PET cracking facility may include at least 55, at least 60, at least 65, at least 70, at least 75, at least 80, at least 85, at least 90, or at least 95 weight percent of waste PET. In the PET cracking facility, at least a portion of the waste PET can be thermally and/or catalytically cracked and the cracked stream may then be separated to form a stream of recycled content aromatics (r-aromatics).

As shown in FIG. 2, at least a portion of the r-benzene stream withdrawn from the aromatics complex can be introduced into at least one chemical processing facility, wherein it can be subjected to one or more chemical processing reactions to provide at least one r-organic chemical compound. The r-benzene stream withdrawn from the aromatics complex can comprise at least 85, at least 90, at least 95, at least 97, or at least 99 weight percent benzene and may or may not include non-recycled content.

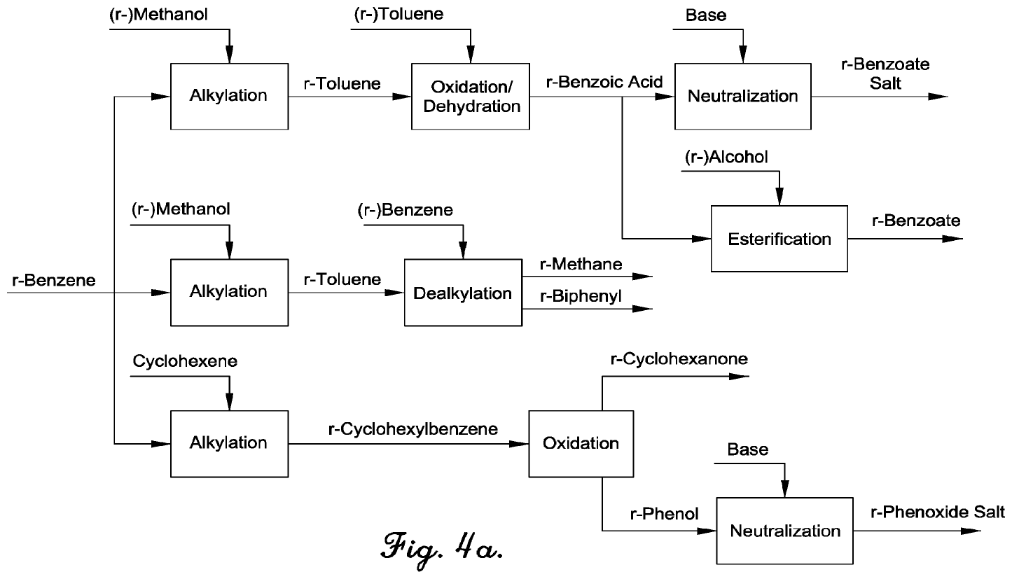

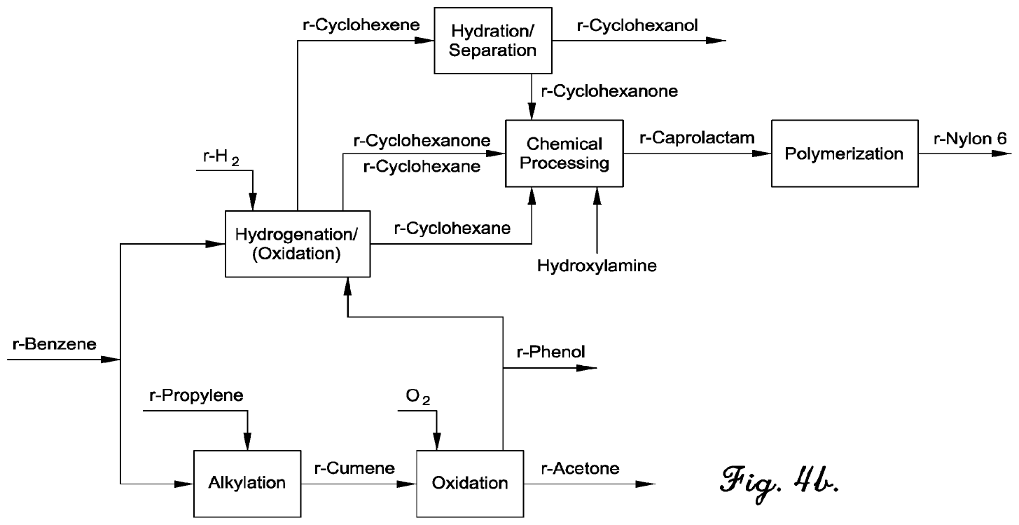

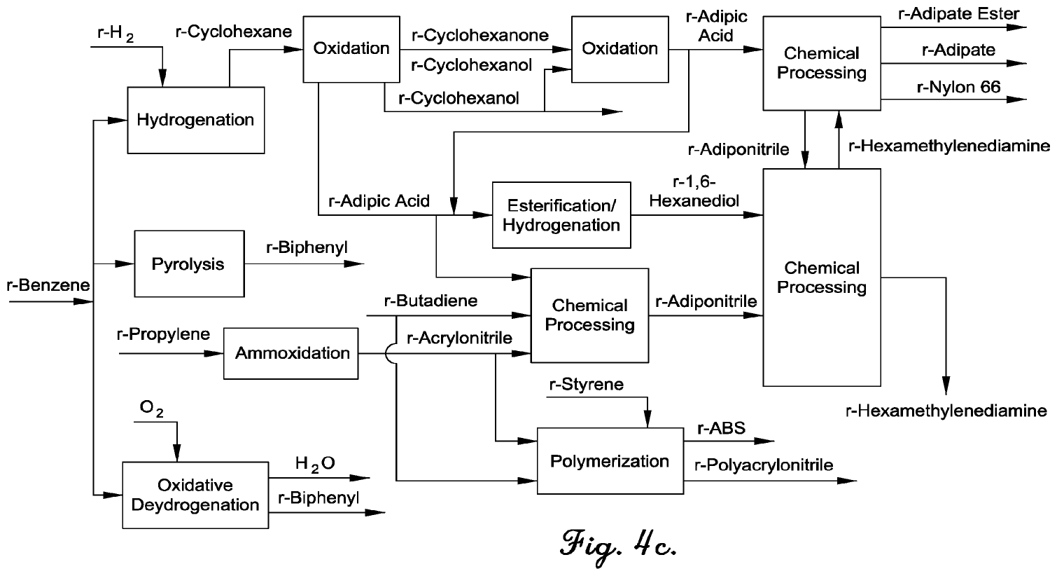

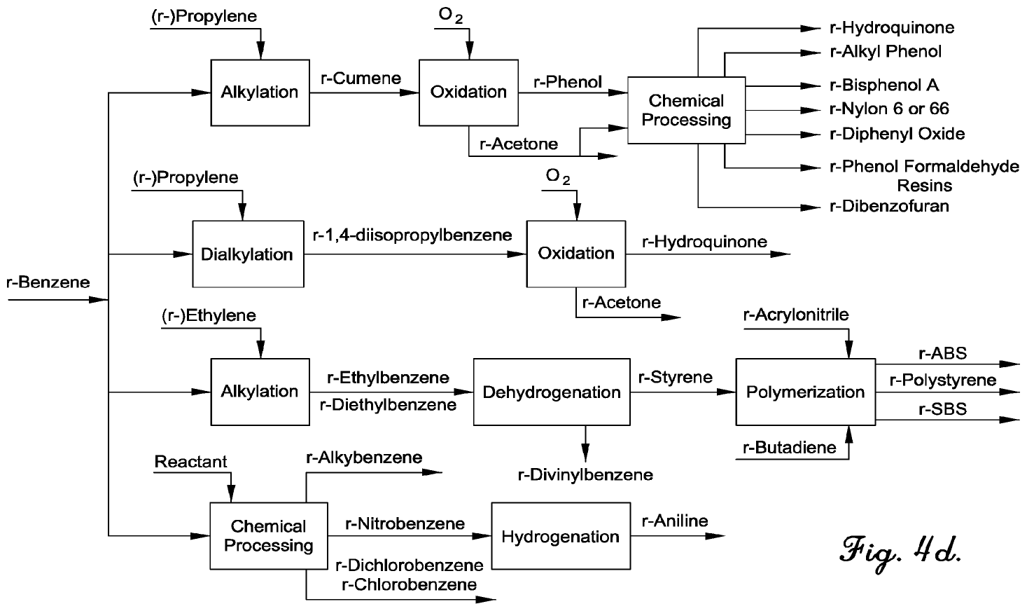

Referring now to FIGS. 4a-d, several block flow diagrams of the main processing steps that can be used in one or more chemical processing facilities for forming a variety of r-organic chemical compounds from r-benzene are provided. The diagrams shown in FIGS. 4a-d provide the basic chemical pathway from r-benzene to several r-organic chemical compounds. Each of these will be described in detail below. Specific processing parameters (e.g., temperature, pressure, residence time, catalyst type and amount, etc.) of each process represented in these FIGS. is not particularly limited and can be any suitable for producing the desired r-organic chemical compound. Additionally, although shown with recycled content feed streams in FIGS. 4a-d, it should be understood that the feed and/or additional reactants (e.g., hydrogen, oxygen, methanol, etc.) to one or more of these chemical processing steps/zones can comprise non-recycled content.

In one embodiment or in combination with any embodiment mentioned herein, at least a portion of the r-benzene from an aromatics complex can be processed in a chemical processing facility to provide recycled content benzoate (r-benzoate) and/or recycled content benzoate salts (r-benzoate salts). As shown in FIG. 4a, this can include alkylating r-benzene with methanol to provide recycled content toluene (r-toluene). The methanol can include recycled content methanol (r-methanol) formed by, for example, processing recycled content syngas (r-syngas) as shown in FIG. 2. In some cases, at least a portion of the methanol for this or any application described herein can include sustainable content methanol (s-methanol) formed by, for example, processing biomass.

Next, at least a portion of the r-toluene can be oxidized and dehydrated to form recycled content benzoic acid (r-benzoic acid), which can either be neutralized with a base (such as a sodium or potassium oxide or hydroxide) to form a recycled content benzoate salt (r-benzoate salt) or esterified with an alcohol, including a recycled content alcohol (r-alcohol) to provide recycled content benzoate. Only one or both of these reactions can take place at a single chemical processing facility.

When the chemical processing facility includes an esterification step/zone, at least a portion of the alcohol (or r-alcohol) reacted with the benzoic acid can be a monoalcohol and the r-benzoate can comprise a recycled content monobenzoate (r-monobenzoate), or the alcohol can be a diol (or polyol) and the r-benzoate can be a recycled content dibenzoate (r-dibenzoate). Examples of suitable alcohols include, but are not limited to, ethylene glycol, triethylene glycol, isodecyl alcohol, isononyl alcohol, dipropylene glycol, diethylene glycol, and neopentyl glycol. Examples of r-monobenzoates and r-dibenzoates can include, but are not limited to, recycled content triethylene glycol dibenzoate (r-triethylene glycol dibenzoate), recycled content isodecyl benzoate (r-isodecyl benzoate), recycled content isononyl benzoate (r-isononyl benzoate), recycled content diproplyene glycol dibenzoate (r-diproplyene glycol dibenzoate), recycled content diethylene glycol dibenzoate (r-diethylene glycol dibenzoate), and recycled content neopentyl glycol dibenzoate (r-neopentyl glycol dibenzoate). One or more of these alcohols and/or organic chemical compounds may also include non-recycled content.

Additionally, or in the alternative, at least a portion of the r-benzene from the aromatics complex can be processed to form r-biphenyl, as shown in FIGS. 4a and 4c. In some cases, the r-biphenyl can be formed by oxidative dehydration of benzene (which produces water), or pyrolysis of benzene optionally in the presence of a catalyst, such as nickel, as shown in FIG. 4c. In other cases, the r-biphenyl can be formed by alkylating at least a portion of the r-benzene with methanol (or r-methanol) to provide r-toluene, and then dealkylating at least a portion of the r-toluene in the presence of benzene (or r-benzene) to form r-biphenyl and r-methane, as shown in FIG. 4a. In some cases (not shown in FIG. 4a), at least a portion of the biphenyl can be alkylated to form a recycled content alkylated biphenyl (r-alkylated biphenyl).

At least a portion of the r-biphenyl can then be further processed by combination with diphenyl oxide or recycled content diphenyl oxide (r-DPO), which can also include recycled content from r-benzene as described with respect to FIG. 4d to form a recycled content heat transfer medium (r-HTM). In some embodiments, the r-HTM can include one or more other components which may or may not include recycled content. For example, the r-HTM may comprise one or more of at least one terphenyl (including meta-terphenyl, para-terphenyl, ortho-terphenyl, and combinations thereof), at least one phenoxybiphenyl (including meta-phenoxybiphenyl, ortho-phenoxybiphenyl, or ortho-phenoxybiphenyl), at least one diphenoxybiphenyl (including meta-diphenoxybiphenyl, ortho-diphenoxybiphenyl, and para-diphenoxybiphenyl), penanthrene, dibenzofuran, naphtalane, and combinations thereof.

In one embodiment or in combination with any embodiment mentioned herein, at least one of the above additional components added to the r-HTM can also include recycled content. For example, any of the terphenyl compounds can comprise recycled content terphenyl (r-terphenyl) compounds and may originate from the pyrolysis of benzene shown in FIG. 4c. Additionally, any of the phenoxybiphenyls and diphenoxy benzenes can comprise a recycled content phenoxybiphenyl and may be formed by, for example, catalytic reaction of recycled content dichlorobenzene (r-dichlorobenzene) with potassium phenate, which may also include recycled content potassium phenate (r-potassium phenate) from, for example, a recycled content phenoxide salt (r-phenoxide salt) formed by reacting r-phenol with a base (e.g., potassium or sodium base), as shown in FIG. 4a. In some cases, r-dichlorobenzene may be formed by chlorination of benzene, as shown in FIG. 4d.

Examples of suitable blends of components used to form r-HTM can include, but are not limited to, r-biphenyl and r-DPO optionally with recycled content polyphenyl ether (r-polyphenyl ether) and partially hydrogenated r-terphenyl and even recycled content quaterphenyl (r-quarterphenyl), both of which can originate from the pyrolysis of r-benzene. In some cases, the hydrogen used to hydrogenate the terphenyl and quarterphenyls can comprise recycled content and in some cases, it may comprise non recycled content.

As shown in FIGS. 4a and 4b, at least a portion of the r-benzene can be processed in the chemical processing step/facility to provide recycled content phenol (r-phenol). In the embodiment shown in FIG. 4a, the r-benzene can be alkylated with cyclohexene to form recycled content cyclohexylbenzene (r-cyclohexylbenzene), which can then be oxidized to form recycled content cyclohexanone (r-cyclohexanone) and recycled content phenol (r-phenol).

Alternatively, as shown in FIG. 4b, at least a portion of the r-benzene can be alkylated with propylene to form recycled content cumene. In some cases, at least a portion of the propylene can be recycled content propylene (r-propylene) and may originate from a refinery and/or steam cracking facility that processes waste plastic or a hydrocarbon stream derived from waste plastic as discussed herein. Turning back to FIG. 4b, at least a portion of the r-cumene can then be oxidized thereby forming recycled content acetone (r-acetone) and recycled content phenol (r-phenol). At least a portion of the r-phenol can be used in further chemical processing steps, as discussed in detail below.

As also shown in FIGS. 4b and 4c, at least a portion of the r-benzene can be processed to form recycled content polyamides, including recycled content nylon 6 (r-nylon 6) and recycled content nylon 66 (r-nylon 66), as well as various precursors of these polymers. For example, turning first to FIG. 4b, at least a portion of the r-benzene from the aromatics complex can be hydrogenated to form recycled content cyclohexane (r-cyclohexane) or recycled content cyclohexene (r-cyclohexene). In some cases, the hydrogen used for this processing step can include recycled content hydrogen (r-hydrogen), and/or it may comprise non recycled content.

As shown in FIG. 4b, at least a portion of the r-cyclohexene can be hydrated (typically in the presence of a zeolite) to form a mixture of recycled content cyclohexanone (r-cyclohexanone) and recycled content cyclohexanol (r-cyclohexanol). Alternatively, or in addition, at least a portion of the r-cyclohexane can be oxidized and/or at least a portion of the r-phenol can be hydrogenated to provide a similar mixture of r-cyclohexanone and r-cyclohexanol (also called ketone-alcohol oil, or KA oil). The r-KA oil stream can then be separated to form a stream of r-cyclohexanol, which can be removed as a chemical product or intermediate, and an intermediate stream of r-cyclohexanone. The r-cyclohexanone can be further reacted with hydroxylamine to form a recycled content oxime intermediate, which can then be further treated with acid to provide recycled content caprolactam (r-caprolactam). In some cases, at least a portion of the r-cyclohexane can be reacted with a catalyst in the presence of UV light to form r-caprolactam. As generally shown in FIG. 4b, at least a portion of the r-caprolactam can be subjected to ring opening polymerization to form recycled content nylon 6 (r-nylon 6).

Turning now to FIG. 4c, at least a portion of the r-benzene can be used to form recycled content nylon 66 (r-nylon 66) and related r-organic chemical compounds. For example, as shown in FIG. 4c, at least a portion of the r-benzene can be hydrogenated to form recycled content cyclohexane (r-cyclohexane). All or a portion of the hydrogen can be recycled content hydrogen and/or the hydrogen can comprise non-recycled content. In one embodiment or in combination with any embodiment mentioned herein, the resulting r-cyclohexane can be oxidized to form a mixture of recycled content cyclohexanol (r-cyclohexanol) and recycled content cyclohexanone (r-cyclohexanone). The r-cyclohexanol can optionally be separated out as a recycled content product stream, or it may be introduced, alone or in combination with the r-cyclohexanone (e.g., as r-KA oil), to further oxidation to form recycled content adipic acid (r-adipic acid). Alternatively, the r-cyclohexane can be oxidized to form a mixture of recycled content adipic acid (r-adipic acid) and recycled content hydroxycaproic acid (r-hydroxycaproic acid) in a single stage or zone.

As shown in FIG. 4c, in some cases, at least a portion of the r-adipic acid can be reacted with a base (e.g., sodium or potassium oxide or hydroxide) to form recycled content adipates (r-adipates) and/or with an alcohol (e.g., a C1 to C18, C2 to C16, or C3 to C12 straight chain or branched alcohol) to provide recycled content adipate esters (r-adipate esters). The alcohol can comprise recycled content alcohol (r-alcohol) and can be selected from the group consisting of, for example, methanol, ethanol, octanol, decanol, isodecanol, and 2-ethylhexanol.

Examples of r-adipates include, but are not limited to, recycled content sodium adipate (r-sodium adipate) and recycled content potassium adipate (r-potassium adipate). Examples of r-adipate esters include, but are not limited to, recycled content bis (2-ethylhexyl) adipate (r-bis(ethylhexyl) adipate), recycled content dioctyl adipate (r-dioctyl adipate), and recycled content dimethyl adipate (r-dimethyl adipate). Some r-adipates can be used as recycled content plasticizers in plasticized polymer compositions.

As also shown in FIG. 4c, the r-adipic acid can be esterified and hydrogenated to form recycled content 1,6-hexanediol (r-1,6-hexanediol), which can then be reacted with ammonia to form recycled content hexamethylenediamine (r-hexamethylenediamine). Alternatively, or in addition, at least a portion of the r-adipic acid can be aminated in the presence of a catalyst to provide recycled content adiponitrile (r-adiponitrile). The r-adiponitrile can also be formed by the ammoxidation of recycled content propylene (r-propylene) to form recycled content acrylonitrile (r-acrylonitrile). Further, recycled content butadiene (r-butadiene) can be reacted with hydrogen cyanide to form r-adiponitrile or it can be oxidized, further reacted in the presence of a cyanide catalyst, and then hydrogenated to form r-adiponitrile. At least a portion of the r-propylene and/or r-butadiene can come from one or more of the conversion facilities, such as the refinery and/or steam cracking facility. The r-adiponitrile can also be formed by electrohydrodimerization of r-acrylonitrile.

In some cases, the r-adiponitrile can be hydrogenated (with recycled and/or non-recycled content hydrogen) to form recycled content hexamethylenediamine (r-hexamethylenediamine). The r-hexamethylenediamine can be withdrawn as a product stream, or it can be reacted with adipic acid (or r-adipic acid) to form recycled content nylon 66 (r-nylon 66).

As shown in FIG. 4c, in one embodiment or in combination with any embodiment mentioned herein, a stream of r-butadiene (or butadiene) and r-acrylonitrile (or acrylonitrile) can be polymerized with styrene (or r-styrene) to form a recycled content ABS polymer (r-ABS). Additionally, or alternatively, the r-acrylonitrile can also be polymerized to form recycled content polyacrylonitrile (r-polyacrylonitrile), or the r-styrene and r-butadiene can be polymerized to form a recycled content SBS polymer (r-SBS).

Turning now to FIG. 4d, additional chemical pathways for converting r-benzene to r-organic chemical compounds are shown. For example, in some cases, r-benzene can be alkylated with propylene (or r-propylene) or ethylene (r-ethylene) or it can be dialkylated with propylene (or r-propylene). At least a portion of the r-propylene and/or r-ethylene can originate from one or more of the conversion facilities (e.g., the steam cracking facility and/or the refinery).

When alkylated with propylene (r-propylene), the r-benzene can be used to form recycled content cumene (r-cumene), which can then be oxidized to form recycled content phenol (r-phenol) and recycled content acetone (r-acetone). The r-phenol and/or r-acetone can be used as intermediates to form a wide variety of r-organic chemical compounds. For example, as shown in FIG. 4d, at least a portion of the r-phenol can be hydroxylated (with hydrogen and/or r-hydrogen) in the presence of a catalyst to form recycled content hydroquinone (r-hydroquinone) and recycled content catechol (r-catechol). Alternatively, at least a portion of the r-phenol can be esterified with at least one alcohol to provide an alkyl substituted phenol (r-alkyl phenol), with an alkyl group including, for example, between 1 and 20 carbon atoms per molecule. The r-alkyl phenol can be selected from the group consisting of recycled content propyl phenol, recycled content butyl phenol, recycled content amyl phenol, recycled content heptyl phenol, recycled content octyl phenol, recycled content nonyl phenol, recycled content dodecyl phenol, and recycled content cresols (ethyl phenol) and recycled content xylenols (methyl phenol). The r-alkyl phenyl can also comprise a recycled content long chain alkyl phenyl (r-LCAP) formed by alkylating at least a portion of the r-phenol with an olefin, and optionally an r-olefin.

Further, in some cases, at least a portion of the r-phenol and r-acetone can be reacted with one another in the presence of a catalyst to provide recycled content bisphenol-A (r-bisphenol-A), which can be used to form several different types of polymers. For example, the r-bisphenol-A can be further polymerized to form recycled content polycarbonate (r-polycarbonate), recycled content epoxy resins (r-epoxy resins), recycled content vinyl ester resins (r-vinyl ester resins), recycled content polycyanurates (r-polycyanurates), recycled content polyetherimides (r-polyetherimides), and polysulfones (r-polysulfones).

Additionally, at least a portion of the r-phenol can be reacted with bromobenzene and catalyst to form recycled content diphenyl oxide (r-diphenyl oxide), which is also called r-diphenyl ether. As discussed previously, the r-diphenyl oxide can be combined with other r-organic chemical compounds (e.g., r-biphenyl) to provide a recycled content heat transfer medium (r-HTM). Further, as shown in FIG. 4d, at least a portion of the r-phenol can be further polymerized with formaldehyde (or recycled content formaldehyde, r-formaldehyde) to form recycled content phenol formaldehyde resins (r-phenol formaldehyde resins). In some cases, at least a portion of the r-formaldehyde can originate from the processing of recycled content syngas (r-syngas) to form recycled content methanol (r-methanol), which can be carbonylated to form r-formaldehyde. The r-phenol may also be reacted over a catalyst and then separated to provide recycled content dibenzofuran (r-dibenzofuran), which may also be used in an r-HTM.

As also shown in FIG. 4d, when dialkylated with propylene (r-propylene), the r-benzene can form recycled content 1,4-diisopropyl benzene (r-1,4-diisopropyl benzene), which can be oxidized to form recycled content hydroquinone (r-hydroquinone) and recycled content acetone. Additionally, when alkylated with ethylene (or r-ethylene), the r-benzene can be used to form recycled content ethylbenzene (r-ethylbenzene), which can be dehydrogenated to form recycled content styrene (r-styrene). The r-styrene can be polymerized by itself to form recycled content polystyrene (r-polystyrene), or it can be polymerized with acrylonitrile (or r-acrylonitrile) and butadiene (r-butadiene) to form r-ABS as discussed previously with respect to FIG. 4c.

Additionally, as shown in FIG. 4d, the alkylation of r-benzene with ethylene (or r-ethylene) may also produce recycled content diethylbenzene, which when dehydrogenated, provides a recycled content divinylbenzene (r-divinylbenzene). In some cases, at least a portion of the r-divinylbenzene may be polymerized with r-styrene (not shown in FIG. 4d) to provide recycled content styrene-divinylbenzene (r-SDVB).

Even further, as shown in FIG. 4d, at least a portion of the r-benzene can be further reacted with one or more different reactants to form recycled content substituted benzenes. For example, the reactant can include an alkylating reactant and the result can be a recycled content alkylbenzene (r-alkylbenzene), such as, for example, recycled content toluene (r-toluene), recycled content ethyl benzene (r-ethyl benzene), recycled content propyl benzene (r-propyl benzene), and recycled content butyl benzene (r-butyl benzene), and isomers of these r-organic chemical compounds. The alkylating reactant can be straight chain or branched and may have a C1 to C18, C2 to C12, or C3 to C10 alkyl group.

Additionally, at least a portion of the r-benzene can be nitrated by reaction with several acidic catalysts, including nitric acid, to provide recycled content nitrobenzene (r-nitrobenzene), which in some cases, can be further hydrogenated to form recycled content aniline (r-aniline). Further, at least a portion of the r-benzene may be halogenated to form a recycled content halogenated benzene (r-halogenated benzene), such as, for example, recycled content chlorobenzene (r-chlorobenzene) and recycled content bromobenzene (r-bromobenzene).

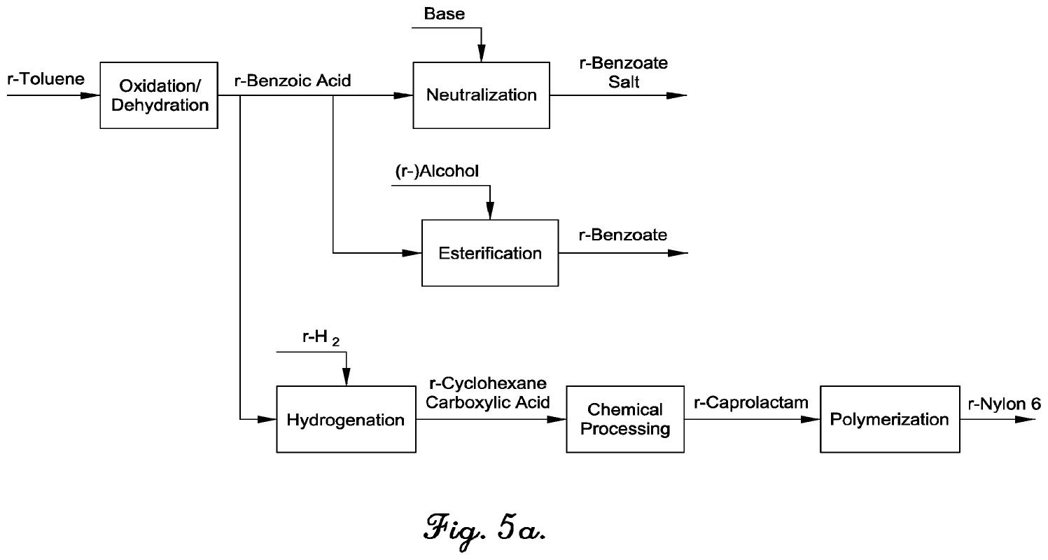

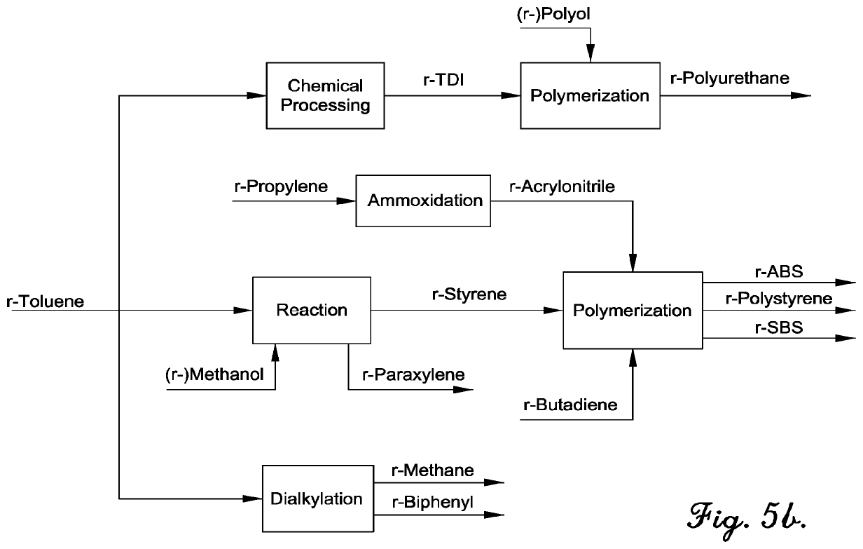

Turning now to FIGS. 5a and 5b, several block flow diagrams of the main processing steps that can be used in one or more chemical processing facilities for forming a variety of r-organic chemical compounds from r-toluene are provided. The diagrams shown in FIGS. 5a and 5b provide the basic chemical pathway from r-toluene to several r-organic chemical compounds. Each of these will be described in detail below. Specific processing parameters (e.g., temperature, pressure, residence time, catalyst type and amount, etc.) of each process represented in these FIGS. is not particularly limited and can be any suitable for producing the desired r-organic chemical compound. Additionally, although shown with recycled content feed streams in FIGS. 5a and 5b, it should be understood that the feed and/or additional reactants (e.g., hydrogen, oxygen, methanol, etc.) to one or more of these chemical processing steps/zones can comprise non-recycled content.

Turning initially to FIG. 5a, at least a portion of the r-toluene can be processed in a chemical processing facility to provide recycled content benzoate (r-benzoate) and/or recycled content benzoate salts (r-benzoate salts). As shown in FIG. 5a, oxidizing and dehydrating at least a portion of the r-toluene to form recycled content benzoic acid (r-benzoic acid), which can either be neutralized with a base (such as a sodium or potassium oxide or hydroxide) to form a recycled content benzoate salt (r-benzoate salt) or esterified with an alcohol, including a recycled content alcohol (r-alcohol) to provide recycled content benzoate. Only one or both of these reactions can take place at a single chemical processing facility.

When the chemical processing facility includes an esterification step/zone, at least a portion of the alcohol (or r-alcohol) reacted with the benzoic acid can be a monoalcohol and the r-benzoate can comprise a recycled content monobenzoate (r-monobenzoate), or the alcohol can be a diol (or polyol) and the r-benzoate can be a recycled content dibenzoate (r-dibenzoate). Examples of suitable alcohols include, but are not limited to, ethylene glycol, triethylene glycol, isodecyl alcohol, isononyl alcohol, dipropylene glycol, diethylene glycol, and neopentyl glycol. Examples of r-monobenzoates and r-dibenzoates can include, but are not limited to, recycled content triethylene glycol dibenzoate (r-triethylene glycol dibenzoate), recycled content isodecyl benzoate (r-isodecyl benzoate), recycled content isononyl benzoate (r-isononyl benzoate), recycled content diproplyene glycol dibenzoate (r-diproplyene glycol dibenzoate), recycled content diethylene glycol dibenzoate (r-diethylene glycol dibenzoate), and recycled content neopentyl glycol dibenzoate (r-neopentyl glycol dibenzoate). One or more of these alcohols and/or organic chemical compounds may also include non-recycled content.

Additionally, as also shown in FIG. 5a, at least a portion of the r-benzoic acid formed by oxidizing r-toluene can be hydrogenated (optionally with recycled content hydrogen) to form recycled content cyclohexane carboxylic acid (r-cyclohexane carboxylic acid), which can be further reacted to form r-caprolactam. As described previously, at least a portion of the r-caprolactam can be polymerized to form recycled content nylon 6 (r-nylon 6).

Turning now to FIG. 5b, at least a portion of the r-toluene can be reacted in a chemical processing step/facility with nitric acid to form recycled content dinitrotoluene (r-dinitrotoluene), which can then be hydrogenated (optionally with r-hydrogen) to form recycled content 2,4-diaminotoluene (r-2,4-diaminotoluene). At least a portion of the r-2,4-diaminotoluene can then be reacted with phosgene to form recycle content toluene diisocyanate (r-TDI). In one embodiment or in combination with any embodiment mentioned herein, at least a portion of the phosgene can comprise recycled content phosgene (r-phosgene) formed by reacting recycled content carbon monoxide (r-CO) with chlorine. At least a portion of the r-CO can originate from the separation of r-syngas formed by the molecular reforming of waste plastic, for example.

As shown in FIG. 5b, at least a portion of the r-TDI can be further reacted with a polyol (including, for example, a recycled content polyol) to form recycled content polyurethane (r-polyurethane). When the polyol includes recycled content, it may originate from one or more monomers used to form the r-polyol, including, but not limited to r-DMT, r-TPA, and r-EG or other r-alcohol. The r-DMT and/or r-TPA can be formed by oxidizing and further processing at least a portion of the r-paraxylene from the same or a different aromatics complex, and the r-EG can be formed from r-ethylene from the steam cracking facility and/or refinery.

In other embodiments illustrated in FIG. 5b, at least a portion of the r-toluene may be reacted with methanol (optionally, r-methanol) to provide recycled content styrene (r-styrene). The r-styrene can be polymerized by itself to form recycled content polystyrene (r-polystyrene), or it can be polymerized with acrylonitrile (or r-acrylonitrile) and butadiene (r-butadiene) to form r-ABS as discussed previously with respect to FIGS. 4c and 4d.

Alternatively, at least a portion of the toluene can be reacted with methanol (and, optionally, r-methanol) to provide recycled content paraxylene (r-paraxylene), which may be further processed as described herein. In some cases, this reaction may be performed within the aromatics complex over an acidic catalyst, preferably on a shape-selective molecular sieve catalyst such as ZSM-5, and the resulting r-paraxylene may be combined with other paraxylene (or r-paraxylene) recovered in the aromatics complex and withdrawn as a single stream, as shown in FIG. 3.

Finally, as also illustrated in FIG. 5b, at least a portion of the r-toluene can be dealkylated to form recycled content methane (r-methane) and recycled content biphenyl (r-biphenyl). As discussed previously, at least a portion of the r-biphenyl can be used to form an r-HTM.

### DEFINITIONS

It should be understood that the following is not intended to be an exclusive list of defined terms. Other definitions may be provided in the foregoing description, such as, for example, when accompanying the use of a defined term in context.

As used here, the term “light gas” refers to a hydrocarbon-containing stream comprising at least 50 weight percent of C4 and lighter hydrocarbon components. Light hydrocarbon gas may include other components such as nitrogen, carbon dioxide, carbon monoxide, and hydrogen, but these are typically present in amount of less than 20, less than 15, less than 10, or less than 5 weight percent, based on the total weight of the stream.

As used herein, the terms “median boiling point” or “T50” refers to the median boiling point of a process stream (i.e., the temperature value where 50 weight percent of the stream composition boils above the temperature value and 50 weight percent of the stream composition boils below the temperature value).

As used herein, the term “boiling point range” or “cut point” refers to the range of temperatures over which a specified petroleum fraction boils. The lower value in a boiling point range is the initial boiling point (IBP) temperature for that specified fraction and the higher value is the end point (EP) temperature for that specified fraction.

As used herein, the term “naphtha” refers to a physical mixture of

hydrocarbon components separated in at least one distillation column of a refining facility that has a boiling point range between 90 to 380° F.

As used herein, the term “light naphtha” refers to a specific portion of a naphtha cut in a refinery having a boiling point range between 90 and 190° F.

As used herein, the term “heavy naphtha” refers to a specific portion of a naphtha cut in a refinery having a boiling point range between 190 and 380° F.

As used herein, the terms “distillate” and “kerosine” refer to a physical mixture of hydrocarbon components separated in at least one distillation column of a refining facility that has a boiling point range greater than 380 to 520° F.

As used herein, the term “hydrocracker distillate” refers to a distillate cut removed from a hydrocracker unit.

As used herein, the term “gas oil” refers to a physical mixture of hydrocarbon components separated in at least one distillation column of a refining facility that has a boiling point range greater than 520 to 1050° F.

As used herein, the term “atmospheric gas oil” refers to a gas oil produced by the atmospheric distillation unit.

As used herein, the term “light gas oil” or “LGO” refers to a specific portion of gas oil cut in a refinery having a boiling point range between greater than 520 and 610° F.

As used herein, “light vacuum gas oil” or “LVGO” refers to a light gas oil produced by the vacuum distillation unit.

As used herein, “light vacuum gas oil” or “LCGO” refers to a light gas oil produced by the coker unit.

As used herein, the term “heavy gas oil” or “HGO” refers to a specific portion of a gas oil cut in a refinery having a boiling point range between greater than 610 and 800° F.

As used herein, “heavy vacuum gas oil” or “HVGO” refers to a heavy gas oil produced by the vacuum distillation unit.

As used herein, “heavy coker gas oil” or “HCGO” refers to a heavy gas oil produced by the coker unit.

As used herein, the term “vacuum gas oil” or “VGO” refers to a specific portion of a gas oil cut in a refinery having a boiling point range between greater than 800 and 1050° F. Vacuum gas oil is separated from the initial crude oil using a vacuum distillation column operated at a pressure below atmospheric pressure.

As used herein, the term “residue” or “resid” refers to the heaviest cut from a distillation column in a refinery and having a boiling point range between greater than 1050° F.

As used herein, the term “vacuum resid” refers to a resid product from the vacuum distillation column.

As used herein, the term “atmospheric resid” refers to a resid product from the atmospheric distillation column.

As used herein, the term “gas plant” refers to equipment, including one or more distillation columns as well as ancillary equipment as well as pumps, compressors, valves, etc. in a refinery for processing a hydrocarbon feed stream comprising predominantly C6 and lighter components to provide one or more purified streams of C1 to C6 alkanes and/or olefins.

As used herein, the term “saturated gas plant” refers to a gas plant in a refinery for processing a hydrocarbon feed stream comprising predominantly saturated hydrocarbons (alkanes). The feed stream to a saturated gas plant includes less than 5 weight percent of olefins, based on the total feed to the plant. The feed to a saturated gas plant in a refinery may come, directly or indirectly, from the crude distillation unit or vacuum distillation unit and may undergo little or no cracking.

As used herein, the term “unsaturated gas plant” refers to a gas plant in a refinery for processing a hydrocarbon feed stream comprising saturated hydrocarbons (alkanes) and unsaturated hydrocarbons (olefins). The feed stream to an unsaturated gas plant includes at least 5 weight percent of olefins, based on the total feed to the plant. The feed to a saturated gas plant in a refinery may come indirectly from the crude unit or vacuum distillation unit and may undergo one or more cracking steps prior to entering the gas plant.

As used herein, the term “gas oil cracker” refers to a cracking unit for processing a feed stream comprising predominantly gas oil and heavier components. Although a gas oil cracker can process lighter components, such as distillate and naphtha, at least 50 weight percent of the total feed to a gas oil cracker includes gas oil and heavier components. Gas oil crackers may be operated at temperatures of at least 350° F., at least 400° F., at least 450° F., at least 500° F., at least 550° F., or at least 600° F. and/or not more than 1200° F., not more than 1150° C., not more than 1100° F., not more than 1050° F., not more than 1000° F., not more than 900° F., or not more than 800° F. Gas oil crackers may be operated at or near atmospheric pressure (e.g., at a pressure of less than 5 psig, less than 2 psig, or 1 psig) or may be operated at elevated pressure (e.g., at a pressure of at least 5 psig, at least 10 psig, at least 25 psig, at least 50 psig, at least 100 psig, at least 250 psig, at least 500 psig, or at least 750 psig.) Additionally, the cracking in gas oil crackers may be carried with or without a catalyst, and it may or may not be conducted in the presence of hydrogen and/or steam.

As used herein, the term “fluidized catalytic cracker” or “FCC” refers to a set of equipment, including a reactor, a regenerator, a main fractionator, as well as ancillary equipment such as pipes, valves, compressors, and pumps, for reducing the molecular weight of a heavy hydrocarbon stream via catalytic cracking in a fluidized catalyst bed.

As used herein, the terms “reformer” or “catalytic reformer” refer to a process or facility in which a feedstock comprising predominantly C6-C10 alkanes is converted to a reformate comprising branched hydrocarbons and/or cyclic hydrocarbons in the presence of a catalyst.

As used herein, the term “reformate” refers to a liquid product stream produced by a catalytic reformer process.

As used herein, the term “hydroprocessing” refers to chemical processing of a hydrocarbon stream with or in the presence of hydrogen. Hydroprocessing is typically a catalytic process and includes hydrocracking and hydrotreating.

As used herein, the term “hydrocracking” refers a type of hydroprocessing where the hydrocarbon molecules are cracked (i.e., undergo a reduction in molecular weight).

As used herein, the term “hydrotreating” refers to a type of hydroprocessing that does not crack the hydrocarbon molecules, but instead removes oxygen, sulfur, and other heteroatoms by hydrogenolysis or to saturate unsaturated bonds by hydrogenation. It may or may not be carried out in the presence of a catalyst.

As used herein, the term “distillation” refers to separation of a mixture of components by boiling point difference.

As used herein, the term “atmospheric distillation” refers to distillation performed at a pressure at or near atmospheric, usually to separate crude oil and/or other streams into specified fractions for further processing.

As used herein, the term “vacuum distillation” refers to distillation performed at a pressure below atmospheric and, usually, at a pressure of less than 100 mm Hg at the top of the column.

As used herein, the term “coking” refers to thermal cracking of heavy hydrocarbons (usually atmospheric or vacuum column bottoms) performed to recover light, more valuable products such as naphtha, distillate, gas oil, and light gas.

As used herein, the term “aromatics complex” refers to a process or facility in which a mixed hydrocarbon feedstock, such as a reformate, is converted into one or more benzene, toluene, and/or xylene (BTX) product streams, such as a paraxylene product stream. The aromatics complex may comprise one or more processing steps, in which one or more components of the reformate are subjected to at least one of a separation step, a transalkylating step, a toluene disproportionation step, and/or an isomerization step. The separation step can comprise one or more of an extraction step, a distillation step, a crystallization step, and/or an adsorption step.

As used herein, the term “raffinate” refers to the aromatics-depleted stream removed from the initial separation step in the aromatics complex. Although most commonly used to refer to a stream withdrawn from an extraction step, the term “raffinate” as used with respect to the aromatics complex can also refer to a stream withdrawn from another type of separation, including, but not limited to, distillation or extractive distillation.

As used herein, the terms “pyrolysis oil” or “pyoil” refers to a composition obtained from pyrolysis that is liquid at 25° C. and 1 atm, absolute.

As used herein, the terms “pyrolysis gas” and “pygas” refer to a composition obtained from pyrolysis that is gaseous at 25° C. and 1 atm, absolute.

As used herein, the term “pyrolysis” refers to thermal decomposition of one or more organic materials at elevated temperatures in an inert (i.e., substantially oxygen free) atmosphere.

As used herein, the term “pyrolysis vapor” refers to the overhead or vapor-phase stream withdrawn from the separator in a pyrolysis facility used to remove r-pyrolysis residue from the r-pyrolysis effluent.

As used herein, the term “pyrolysis effluent” refers to the outlet stream withdrawn from the pyrolysis reactor in a pyrolysis facility.

As used herein, the term “r-pyrolysis residue” refers to a composition obtained from waste plastic pyrolysis that comprises predominantly pyrolysis char and pyrolysis heavy waxes.

As used herein, the term “pyrolysis char” refers to a carbon-containing composition obtained from pyrolysis that is solid at 200°° C. and 1 atm, absolute.

As used herein, the term “pyrolysis heavy waxes” refers to C20+ hydrocarbons obtained from pyrolysis that are not pyrolysis char, pyrolysis gas, or pyrolysis oil.

As used herein, the term “pyrolysis gasoline” refers to a hydrocarbon stream of predominantly C5 and heavier components removed from a quench section of a steam cracking facility. Typically, pyrolysis gasoline includes at least 10 weight percent of C6 to C9 aromatics.

As used herein, the term “lighter” refers to a hydrocarbon component or fraction having a lower boiling point than another hydrocarbon component or fraction.

As used herein, the term “heavier” refers to a hydrocarbon component or fraction having a higher boiling point than another hydrocarbon component or fraction.

As used herein, the term “upstream” refers to an item of facility that is positioned prior to another item or facility in a given process flow and may include intervening items and/or facilities.

As used herein, the term “downstream” refers to an item or facility that is positioned after another item or facility in a given process flow and may include intervening items and/or facilities.

As used herein, the term “alkane” refers to a saturated hydrocarbon including no carbon-carbon double bonds.

As used herein, the term “olefin” refers to an at least partially unsaturated hydrocarbon including at least one carbon-carbon double bond.

As used herein, the terms “Cx” or “Cx hydrocarbon” or “Cx component” refers to a hydrocarbon compound including “x” total carbons per molecule, and encompasses all olefins, paraffins, aromatics, heterocyclic, and isomers having that number of carbon atoms. For example, each of normal, iso, and tert-butane and butene and butadiene molecules would fall under the general description “C4” or “C4 components.”

As used herein, the terms “r-paraxylene” or “r-pX” refer to being or comprising a paraxylene product that is directly and/or indirectly derived from waste plastic.

As used herein, the term “cracking” refers to breaking down complex organic molecules into simpler molecules by the breaking of carbon-carbon bonds.

As used herein, the term “steam cracking” refers to thermal cracking of hydrocarbons in the presence of steam, usually performed in a furnace of the steam cracking facility.

As used herein, the term “co-located” refers to the characteristic of at least two objects being situated on a common physical site, and/or within five miles of each other, measured as a straight-line distance between two designated points.

As used herein, the term “commercial scale facility” refers to a facility having an average annual feed rate of at least 500 pounds per hour, averaged over one year.

As used herein, the terms “crude” and “crude oil” refer to a mixture of hydrocarbons that exists in liquid phase and is derived from natural underground reservoirs.

As used herein, the terms “recycle content” and “r-content” refer to being or comprising a composition that is directly and/or indirectly derived from waste plastic.

As used herein, the term “predominantly” means more than 50 percent by weight. For example, a predominantly propane stream, composition, feedstock, or product is a stream, composition, feedstock, or product that contains more than 50 weight percent propane.

As used herein, the term “waste material” refers to used, scrap, and/or discarded material.

As used herein, the terms “waste plastic” and “plastic waste” refer to used, scrap, and/or discarded plastic materials.

As used herein, the terms “mixed plastic waste” and “MPW” refer to a mixture of at least two types of waste plastics including, but not limited to the following plastic types: polyethylene terephthalate (PET), one or more polyolefins (PO), and polyvinylchloride (PVC).

As used herein, the term “fluid communication” refers to the direct or indirect fluid connection between two or more processing, storage, or transportation facilities or zones. As used herein, the terms “a,” “an,” and “the” mean one or more.

As used herein, the term “and/or,” when used in a list of two or more items, means that any one of the listed items can be employed by itself or any combination of two or more of the listed items can be employed. For example, if a composition is described as containing components A, B, and/or C, the composition can contain A alone; B alone; C alone; A and B in combination; A and C in combination, B and C in combination; or A, B, and C in combination.

As used herein, the phrase “at least a portion” includes at least a portion and up to and including the entire amount or time period.

As used herein, the term “chemical recycling” refers to a waste plastic recycling process that includes a step of chemically converting waste plastic polymers into lower molecular weight polymers, oligomers, monomers, and/or non-polymeric molecules (e.g., hydrogen, carbon monoxide, methane, ethane, propane, ethylene, and propylene) that are useful by themselves and/or are useful as feedstocks to another chemical production process(es).

As used herein, the terms “comprising,” “comprises,” and “comprise” are open-ended transition terms used to transition from a subject recited before the term to one or more elements recited after the term, where the element or elements listed after the transition term are not necessarily the only elements that make up the subject.

As used herein, the term “cracking” refers to breaking down complex organic molecules into simpler molecules by the breaking of carbon-carbon bonds.

As used herein, the terms “including,” “include,” and “included” have the same open-ended meaning as “comprising,” “comprises,” and “comprise” provided above.

As used herein, the term “predominantly” means more than 50percent by weight. For example, a predominantly propane stream, composition, feedstock, or product is a stream, composition, feedstock, or product that contains more than 50 weight percent propane.

As used herein, the term “chemical pathway” refers to the chemical processing step or steps (e.g., chemical reactions, physical separations, etc.) between an input material and a product, where the input material is used to make the product.

As used herein, the terms “credit-based recycled content,” “non-physical recycled content,” and “indirect recycled content” all refer to matter that is not physically traceable back to a waste material, but to which a recycled content credit has been attributed.

As used herein, the term “directly derived” refers to having at least one physical component originating from waste material.

As used herein, the term “indirectly derived” refers to having an applied recycled content (i) that is attributable to waste material, but (ii) that is not based on having a physical component originating from waste material.

As used herein, the term “located remotely” refers to a distance of at least 0.1, 0.5, 1, 5, 10, 50, 100, 500, or 1000 miles between two facilities, sites, or reactors.

As used herein, the term “mass balance” refers to a method of tracing recycled content based on the mass of the recycled content in a product.

As used herein, the terms “physical recycled content” and “direct recycled content” both refer to matter physically present in a product and that is physically traceable back to a waste material.

As used herein, the term “recycled content” refers to being or comprising a composition that is directly and/or indirectly derived from recycle waste material. Recycled content is used generically to refer to both physical recycled content and credit-based recycled content. Recycled content is also used as an adjective to describe a product having physical recycled content and/or credit-based recycled content.

As used herein, the term “recycled content credit” refers to a non-physical measure of recycled content obtained from a mass of waste plastic that can be directly or indirectly (i.e., via a digital inventory) attributed to a product second material.

As used herein, the term “total recycled content” refers to the cumulative amount of physical recycled content and credit-based recycled content from all sources.

As used herein, the term “waste material” refers to used, scrap, and/or discarded material.

As used herein, the terms “waste plastic” and “plastic waste” refer to used, scrap, and/or discarded plastic materials, including post industrial or pre-consumer waste plastic and post-consumer waste plastic.

As used herein, the term “hydroprocessing unit” refers to a set of equipment, including reaction vessels, a drier, and a main fractionator, as well as ancillary equipment such as pipes, valves, compressors, and pumps, for chemically processing a hydrocarbon stream in the presence of hydrogen. Specific examples of hydroprocessing units include a hydrocracker (or hydrocracking unit) configured to carry out a hydrocracking process and a hydrotreater (or hydrotreating unit) configured to carry out a hydrotreating process.

As used herein, the term “coker” or “coking unit” refers to a set of equipment, including reaction vessels, a drier, and a main fractionator, as well as ancillary equipment such as pipes, valves, compressors, and pumps, for reducing the molecular weight of a heavy hydrocarbon stream via thermal cracking or coking.

As used herein, the terms “steam cracking facility” or “steam cracker” refer to all of the equipment needed to carry out the processing steps for thermally cracking a hydrocarbon feed stream in the presence of steam to form one or more cracked hydrocarbon products. Examples include, but are not limited to, olefins such as ethylene and propylene. The facility may include, for example, a steam cracking furnace, cooling equipment, compression equipment, separation equipment, as well as the pipes, valves, tanks, pumps, etc. needed to carry out the processing steps.

As used herein, the terms “refinery,” “refining facility,” and “petroleum refinery,” refer to all of the equipment needed to carry out the processing steps for separating and converting petroleum crude oil into multiple hydrocarbon fractions, one or more of which can be used as a fuel source, lube oil, bitumen, coke, and as an intermediate for other chemical products.” The facility may include, for example, separation equipment, thermal or catalytic cracking equipment, chemical reactors, and product blending equipment, as well as the pipes, valves, tanks, pumps, etc. needed to carry out the processing steps.

As used herein, the term “pyrolysis facility,” refers to all of the equipment needed to carry out the processing steps for pyrolyzing a hydrocarbon-containing feed stream, which can include or be waste plastic. The facility may include, for example, reactors, cooling equipment, and separation equipment, as well as the pipes, valves, tanks, pumps, etc. needed to carry out the processing steps.

As used herein, the term “terephthalic acid production facility,” or “TPA production facility,” refers to all of the equipment needed to carry out the processing steps for forming terephthalic acid from paraxylene. The facility may include, for example, reactors, separators, cooling equipment, separation equipment such as filters or crystallizers, as well as the pipes, valves, tanks, pumps, etc. needed to carry out the processing steps.

As used herein, the term “polyethylene terephthalate production facility,” or “PET production facility,” refers to all of the equipment needed to carry out the processing steps for forming polyethylene terephthalate (PET) from a terephthalate, ethylene glycol, and, optionally, one or more additional monomers. The facility may include, for example, polymerization reactors, cooling equipment, and equipment to recover solidified and/or pelletized PET, as well as the pipes, valves, tanks, pumps, etc. needed to carry out the processing steps.

As used herein, the term “chemical pathway” refers to the chemical processing step or steps (e.g., chemical reactions, physical separations, etc.) between an input material and a product, where the input material is used to make the product.

As used herein, the terms “credit-based recycled content,” “non-physical recycled content,” and “indirect recycled content” all refer to matter that is not physically traceable back to a waste material, but to which a recycled content credit has been attributed.

As used herein, the term “directly derived” refers to having at least one physical component originating from waste material.

As used herein, the term “indirectly derived” refers to having an applied recycled content (i) that is attributable to waste material, but (ii) that is not based on having a physical component originating from waste material.

As used herein, the term “located remotely” refers to a distance of at least 0.1, 0.5, 1, 5, 10, 50, 100, 500, or 1000 miles between two facilities, sites, or reactors.

As used herein, the term “mass balance” refers to a method of tracing recycled content based on the mass of the recycled content in a product.

As used herein, the terms “physical recycled content” and “direct recycled content” both refer to matter physically present in a product and that is physically traceable back to a waste material.

As used herein, the term “recycled content” refers to being or comprising a composition that is directly and/or indirectly derived from recycle waste material. Recycled content is used generically to refer to both physical recycled content and credit-based recycled content. Recycled content is also used as an adjective to describe a product having physical recycled content and/or credit-based recycled content.

As used herein, the term “recycled content credit” refers to a non-physical measure of recycled content obtained from a mass of waste plastic that can be directly or indirectly (i.e., via a digital inventory) attributed to a product second material.

As used herein, the term “total recycled content” refers to the cumulative amount of physical recycled content and credit-based recycled content from all sources.

As used herein, the term “waste material” refers to used, scrap, and/or discarded material.

As used herein, the terms “waste plastic” and “plastic waste” refer to used, scrap, and/or discarded plastic materials, including post industrial or pre-consumer waste plastic and post-consumer waste plastic.

As used herein, the term “hydroprocessing unit” refers to a set of equipment, including reaction vessels, a drier, and a main fractionator, as well as ancillary equipment such as pipes, valves, compressors, and pumps, for chemically processing a hydrocarbon stream in the presence of hydrogen. Specific examples of hydroprocessing units include a hydrocracker (or hydrocracking unit) configured to carry out a hydrocracking process and a hydrotreater (or hydrotreating unit) configured to carry out a hydrotreating process.

As used herein, the term “coker” or “coking unit” refers to a set of equipment, including reaction vessels, a drier, and a main fractionator, as well as ancillary equipment such as pipes, valves, compressors, and pumps, for reducing the molecular weight of a heavy hydrocarbon stream via thermal cracking or coking.

As used herein, the terms “steam cracking facility” or “steam cracker” refer to all of the equipment needed to carry out the processing steps for thermally cracking a hydrocarbon feed stream in the presence of steam to form one or more cracked hydrocarbon products. Examples include, but are not limited to, olefins such as ethylene and propylene. The facility may include, for example, a steam cracking furnace, cooling equipment, compression equipment, separation equipment, as well as the pipes, valves, tanks, pumps, etc. needed to carry out the processing steps.

As used herein, the terms “refinery,” “refining facility,” and “petroleum refinery,” refer to all of the equipment needed to carry out the processing steps for separating and converting petroleum crude oil into multiple hydrocarbon fractions, one or more of which can be used as a fuel source, lube oil, bitumen, coke, and as an intermediate for other chemical products.” The facility may include, for example, separation equipment, thermal or catalytic cracking equipment, chemical reactors, and product blending equipment, as well as the pipes, valves, tanks, pumps, etc. needed to carry out the processing steps.

As used herein, the term “pyrolysis facility,” refers to all of the equipment needed to carry out the processing steps for pyrolyzing a hydrocarbon-containing feed stream, which can include or be waste plastic. The facility may include, for example, reactors, cooling equipment, and separation equipment, as well as the pipes, valves, tanks, pumps, etc. needed to carry out the processing steps.

As used herein, the term “terephthalic acid production facility,” or “TPA production facility,” refers to all of the equipment needed to carry out the processing steps for forming terephthalic acid from paraxylene. The facility may include, for example, reactors, separators, cooling equipment, separation equipment such as filters or crystallizers, as well as the pipes, valves, tanks, pumps, etc. needed to carry out the processing steps.

As used herein, the term “polyethylene terephthalate production facility,” or “PET production facility,” refers to all of the equipment needed to carry out the processing steps for forming polyethylene terephthalate (PET) from a terephthalate, ethylene glycol, and, optionally, one or more additional monomers. The facility may include, for example, polymerization reactors, cooling equipment, and equipment to recover solidified and/or pelletized PET, as well as the pipes, valves, tanks, pumps, etc. needed to carry out the processing steps.

As used herein the term “chemical processing facility,” refers to all of the equipment needed to carry out the processing steps for one or more chemical processes for converting a starting material to a final chemical product. The facility may include, for example, separation or treatment equipment, reaction equipment, and equipment to recover a final product, as well as the pipes, valves, tanks, pumps, etc. needed to carry out the processing steps.

### CLAIMS NOT LIMITED TO DISCLOSED EMBODIMENTS

The preferred forms of the invention described above are to be used as illustration only and should not be used in a limiting sense to interpret the scope of the present invention. Modifications to the exemplary embodiments, set forth above, could be readily made by those skilled in the art without departing from the spirit of the present invention.

The inventors hereby state their intent to rely on the Doctrine of Equivalents to determine and assess the reasonably fair scope of the present invention as it pertains to any apparatus not materially departing from but outside the literal scope of the invention as set forth in the following claims.

1. A process for producing a recycled content organic chemical compound (r-organic chemical compound), the process comprising:
(a) processing at least a portion of a recycled content C6 to C10 aromatics (r-C6 to C10 aromatics) stream in an aromatics facility to provide recycled content benzene (r-benzene) stream and a recycled content toluene (r-toluene) stream; and
(b) reacting the r-benzene and/or r-toluene in another chemical processing facility to form at least one r-organic chemical compound.

2. The process of claim 1, wherein the reacting includes
(i) oxidizing and dehydrating a toluene feed stream comprising at least a portion of the r-toluene to provide recycled content benzoic acid (r-benzoic acid), or
(ii) neutralizing at least a portion of the benzoic acid with at least one base to provide a recycled content benzoate salt (r-benzoate salt) and/or esterifying at least a portion of the r-benzoic acid with at least one alcohol to provide a recycled content benzoate (r-benzoate), or
(iii) forming recycled content biphenyl (r-biphenyl) from at least a portion of the r-benzene and/or r-toluene, or
(iv) forming recycled content styrene from at least a portion of the r-benzene and/or r-toluene, and further comprising polymerizing at least a portion of the r-styrene to form one or more of recycled content polystyrene (r-polystyrene), recycled content ABS (r-ABS), and recycled content SBS (r-SBS), or
(v) alkylating at least a portion of the r-benzene to form recycled content cumene (r-cumene) and further reacting at least a portion of the r-cumene to provide recycled content phenol (r-phenol) and recycled content acetone (r-acetone), or
(vi) hydrogenating at least a portion of the r-benzene to form recycled content cyclohexane (r-cyclohexane), or
(vii) alkylating the r-benzene with propylene to provide recycled content cumene (r-cumene) and oxidizing at least a portion of the r-cumene to provide recycled content phenol, or
(viii) reacting at least a portion of the r-toluene with nitric acid to provide recycled content dinitrotoluene (r-dinitrotoluene) and hydrogenating at least a portion of the r-dinitrotoluene to provide recycled content 2,4-diaminotoluene (r-2,4-diaminotoluene) and reacting at least a portion of the r-2,4-diaminotoluene with phosgene to provide recycled content toluene diisocyanate (r-TDI).

3. The process of claim 2, further comprising processing at least a portion of the r-phenol and/or r-acetone to provide one or more of the following recycled content organic chemical compounds: recycled content hydroquinone (r-hydroquinone), recycled content alkyl phenol (r-alkyl phenol), recycled content bisphenol-A (r-bisphenol-A), recycled content diphenyl oxide (r-diphenyl oxide) and recycled content phenol formaldehyde resins (r-phenol formaldehyde resins).

4. The process of claim 2, further comprising processing at least a portion of the r-cyclohexane to provide recycled content adipic acid (r-adipic acid) and further comprising polymerizing at least a portion of the r-adipic acid to provide recycled content nylon 66 (r-nylon 66).

5. The process of claim 2, further comprising processing at least a portion of the r-cyclohexane to provide recycled content caprolactam (r-caprolactam) and further comprising polymerizing at least a portion of the r-caprolactam to provide recycled content nylon 6 (r-nylon 6).

6. The process of claim 2, further comprising reacting at least a portion of the r-TDI with at least one polyol to provide recycled content polyurethane (r-polyurethane).

7. The process of claim 1, wherein the C6 to C10 aromatics stream comes from at least one conversion facility selected from the group consisting of a pyrolysis facility, a refinery, a steam cracking facility, and a molecular reforming facility, wherein each conversion facility is configured to process waste plastic or a hydrocarbon stream derived from waste plastic.

8. The process of claim 7, wherein the C6 to C10 aromatics stream comprises at least one of a recycled content reformate (r-reformate) stream from a refinery, a recycled content pyrolysis gasoline (r-pyrolysis gasoline) stream from a steam cracking facility, or a recycled content aromatics (r-aromatics) stream from a methanol-to-aromatics facility.

9. The process of claim 1, wherein the processing of the aromatics complex includes one or more of distillation, extraction, crystallization, adsorption, and combinations thereof), isomerization, alkylation, and transalkylation/disproportionation; and providing a stream of recycled content paraxylene (r-paraxylene) from the aromatics complex.

10. The process of claim 1, wherein at least a portion of the recycled content is physical recycled content.

11. The process of claim 1, wherein at least a portion of the recycled content is credit-based recycled content.

12. A process for producing a recycled content organic chemical compound (r-organic chemical compound), the process comprising: reacting at stream comprising recycled content toluene (r-toluene) and/or a stream comprising recycled content benzene (r-benzene) in at least one chemical processing facility to provide at least one r-organic chemical compound, wherein at least a portion of the r-toluene and/or r-benzene is obtained by processing a recycled content C6 to C10 aromatics (r-C6 to C10 aromatics) stream in an aromatics complex and wherein at least a portion of the r-C6 to C10 aromatics is obtained by processing a recycled content hydrocarbon stream in a steam cracking facility and/or in a reformer facility.

13. The process of claim 12, wherein the reacting includes
(i) oxidizing and dehydrating a toluene feed stream comprising at least a portion of the r-toluene to provide recycled content benzoic acid (r-benzoic acid), or
(ii) neutralizing at least a portion of the benzoic acid with at least one base to provide a recycled content benzoate salt (r-benzoate salt) and/or esterifying at least a portion of the r-benzoic acid with at least one alcohol to provide a recycled content benzoate (r-benzoate), or
(iii) forming recycled content biphenyl (r-biphenyl) from at least a portion of the r-benzene and/or r-toluene, or
(iv) forming recycled content styrene from at least a portion of the r-benzene and/or r-toluene, and further comprising polymerizing at least a portion of the r-styrene to form one or more of recycled content polystyrene (r-polystyrene), recycled content ABS (r-ABS), and recycled content SBS (r-SBS), or
(v) alkylating at least a portion of the r-benzene to form recycled content cumene (r-cumene) and further reacting at least a portion of the r-cumene to provide recycled content phenol (r-phenol) and recycled content acetone (r-acetone), or
(vi) hydrogenating at least a portion of the r-benzene to form recycled content cyclohexane (r-cyclohexane), or
(vii) alkylating the r-benzene with propylene to provide recycled content cumene (r-cumene) and oxidizing at least a portion of the r-cumene to provide recycled content phenol, or
(viii) reacting at least a portion of the r-toluene with nitric acid to provide recycled content dinitrotoluene (r-dinitrotoluene) and hydrogenating at least a portion of the r-dinitrotoluene to provide recycled content 2,4-diaminotoluene (r-2,4-diaminotoluene) and reacting at least a portion of the r-2,4-diaminotoluene with phosgene to provide recycled content toluene diisocyanate (r-TDI).

14. The process of claim 13, further comprising processing at least a portion of the r-phenol and/or r-acetone to provide one or more of the following recycled content organic chemical compounds: recycled content hydroquinone (r-hydroquinone), recycled content alkyl phenol (r-alkyl phenol), recycled content bisphenol-A (r-bisphenol-A), recycled content diphenyl oxide (r-diphenyl oxide) and recycled content phenol formaldehyde resins (r-phenol formaldehyde resins).

15. The process of claim 2, further comprising processing at least a portion of the r-cyclohexane to provide recycled content adipic acid (r-adipic acid) and further comprising polymerizing at least a portion of the r-adipic acid to provide recycled content nylon 66 (r-nylon 66), or processing at least a portion of the r-cyclohexane to provide recycled content caprolactam (r-caprolactam) and further comprising polymerizing at least a portion of the r-caprolactam to provide recycled content nylon 6 (r-nylon 6), or reacting at least a portion of the r-TDI with at least one polyol to provide recycled content polyurethane (r-polyurethane).

16. A process for producing a recycled content organic chemical compound (r-organic chemical compound), the process comprising:
(a) pyrolyzing waste plastic in a pyrolysis facility to provide a recycled content pyrolysis stream (r-pyrolysis stream);
(b) introducing at least a portion of the r-pyrolysis stream into a steam cracking facility to provide a recycled content pyrolysis gasoline (r-pyrolysis gasoline);
(c) processing at least a portion of the r-pyrolysis gasoline in an aromatics complex to provide recycled content benzene (r-benzene) and/or toluene (r-toluene); and
(d) reacting the r-benzene and/or r-toluene in another chemical processing facility to form at least one r-organic chemical compound.

17. The process of claim 16, wherein the introducing includes introducing recycled content pyrolysis oil (r-pyoil) into the inlet of a cracker furnace.

18. A process for producing a recycled content organic chemical compound (r-organic chemical compound), the process comprising:
(a) pyrolyzing waste plastic in a pyrolysis facility to provide a recycled content pyrolysis stream (r-pyrolysis stream);
(b) processing at least a portion of the r-pyrolysis stream in a reformer unit of a refinery to provide a recycled content reformate (r-reformate) stream;
(c) processing at least a portion of the r-reformate stream in an aromatics complex to provide recycled content benzene (r-benzene) and/or toluene (r-toluene); and
(d) reacting the r-benzene and/or r-toluene in another chemical processing facility to form at least one r-organic chemical compound.

19. The process of claim 18, further comprising separating at least a portion of the r-pyrolysis stream in at least one distillation column to provide a recycled content hydrocarbon fraction (r-hydrocarbon fraction) and processing at least a portion of the r-hydrocarbon fraction in the reformer unit.

20. The process of claim 18, further comprising thermally and/or catalytically cracking at least a portion of the r-pyrolysis stream in a heavy oil cracker to provide a recycled content cracked naphtha (r-naphtha) and processing at least a portion of the r-cracked naphtha in the reformer unit.
